SEQUENCE LISTING (58 sequences)

SEQ ID NO: 1 - protein, 117 aa, synthetic construct
QVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSS

SEQ ID NO: 2 - protein, 10 aa, synthetic construct
GSSLTSYGVH

SEQ ID NO: 3 - protein, 16 aa, synthetic construct
VIWPGGSTNYNSALMS

SEQ ID NO: 4 - protein, 9 aa, synthetic construct
VTGTWYFDV

SEQ ID NO: 5 - protein, 107 aa, synthetic construct
DIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIK

SEQ ID NO: 6 - protein, 11 aa, synthetic construct
SASQGISNYLN

SEQ ID NO: 7 - protein, 7 aa, synthetic construct
YTSTLHS

SEQ ID NO: 8 - protein, 9 aa, synthetic construct
QQYSKLPWT

SEQ ID NO: 9 - protein, 124 aa, synthetic construct
EVQLVESGGGLVQPGGSLRLSCAASGFTFNTNAMNWVRQAPGKGLEWVGRIRTKSNNYATYYAESVRDRFTISRDDSKNSLYLQMNSLKTEDTAMYYCVRGGYGIRGEYTMDYWGQGTLVTVSS

SEQ ID NO: 10 - protein, 10 aa, synthetic construct
GFTFNTNAMN

SEQ ID NO: 11 - protein, 19 aa, synthetic construct
RIRTKSNNYATYYAESVRD

SEQ ID NO: 12 - protein, 13 aa, synthetic construct
GGYGIRGEYTMDY

SEQ ID NO: 13 - protein, 112 aa, synthetic construct
DIVMTQSPPTLSLSPGERATLSCRSSKSLLHSNGNTYLYWFQQKPGQSPQLLIYRMSNLASGIPDRFSGSGSGTDFTLTISRLEPEDFAVYYCMQHLEYPFTFGQGTKLEIK

SEQ ID NO: 14 - protein, 16 aa, synthetic construct
RSSKSLLHSNGNTYLY

SEQ ID NO: 15 - protein, 7 aa, synthetic construct
RMSNLAS

SEQ ID NO: 16 - protein, 9 aa, synthetic construct
MQHLEYPFT

SEQ ID NO: 17 - protein, 118 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSS

SEQ ID NO: 18 - protein, 10 aa, synthetic construct
GYSITDYLLH

SEQ ID NO: 19 - protein, 20 aa, synthetic construct
WMGWIDPEDAKTKYGQKFQS

SEQ ID NO: 20 - protein, 9 aa, synthetic construct
NSGTGTMDA

SEQ ID NO: 21 - protein, 107 aa, synthetic construct
DIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 22 - protein, 11 aa, synthetic construct
KASQNVGSNVD

SEQ ID NO: 23 - protein, 7 aa, synthetic construct
KASIRYT

SEQ ID NO: 24 - protein, 9 aa, synthetic construct
MQSKSYPIT

SEQ ID NO: 25 - protein, 123 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 26 - protein, 10 aa, synthetic construct
GSRLDYYAVA

SEQ ID NO: 27 - protein, 17 aa, synthetic construct
CITYSGGSTNYAESVKG

SEQ ID NO: 28 - protein, 14 aa, synthetic construct
EPYGSSCPLSGYDT

SEQ ID NO: 29 - protein, 107 aa, synthetic construct [sequence omitted]

SEQ ID NO: 30 - protein, 214 aa, synthetic construct
DIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 464 aa, synthetic construct
DIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 32 - protein, 469 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIKGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 347 aa, synthetic construct
DIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 34 - protein, 352 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 702 aa, synthetic construct
QVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 36 - protein, 447 aa, synthetic construct
QVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 702 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIKGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 585 aa, synthetic construct
QVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 39 - protein, 585 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 582 aa, synthetic construct
QVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 41 - protein, 582 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 219 aa, synthetic construct
DIVMTQSPPTLSLSPGERATLSCRSSKSLLHSNGNTYLYWFQQKPGQSPQLLIYRMSNLASGIPDRFSGSGSGTDFTLTISRLEPEDFAVYYCMQHLEYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 469 aa, synthetic construct
DIVMTQSPPTLSLSPGERATLSCRSSKSLLHSNGNTYLYWFQQKPGQSPQLLIYRMSNLASGIPDRFSGSGSGTDFTLTISRLEPEDFAVYYCMQHLEYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 44 - protein, 474 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIKGGGGSGGGGSGGGGSDIVMTQSPPTLSLSPGERATLSCRSSKSLLHSNGNTYLYWFQQKPGQSPQLLIYRMSNLASGIPDRFSGSGSGTDFTLTISRLEPEDFAVYYCMQHLEYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 352 aa, synthetic construct
DIVMTQSPPTLSLSPGERATLSCRSSKSLLHSNGNTYLYWFQQKPGQSPQLLIYRMSNLASGIPDRFSGSGSGTDFTLTISRLEPEDFAVYYCMQHLEYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 46 - protein, 357 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSGGGGSGGGGSGGGGSDIVMTQSPPTLSLSPGERATLSCRSSKSLLHSNGNTYLYWFQQKPGQSPQLLIYRMSNLASGIPDRFSGSGSGTDFTLTISRLEPEDFAVYYCMQHLEYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 454 aa, synthetic construct
EVQLVESGGGLVQPGGSLRLSCAASGFTFNTNAMNWVRQAPGKGLEWVGRIRTKSNNYATYYAESVRDRFTISRDDSKNSLYLQMNSLKTEDTAMYYCVRGGYGIRGEYTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 709 aa, synthetic construct
EVQLVESGGGLVQPGGSLRLSCAASGFTFNTNAMNWVRQAPGKGLEWVGRIRTKSNNYATYYAESVRDRFTISRDDSKNSLYLQMNSLKTEDTAMYYCVRGGYGIRGEYTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 49 - protein, 709 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIKGGGGSGGGGSGGGGSEVQLVESGGGLVQPGGSLRLSCAASGFTFNTNAMNWVRQAPGKGLEWVGRIRTKSNNYATYYAESVRDRFTISRDDSKNSLYLQMNSLKTEDTAMYYCVRGGYGIRGEYTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 592 aa, synthetic construct
EVQLVESGGGLVQPGGSLRLSCAASGFTFNTNAMNWVRQAPGKGLEWVGRIRTKSNNYATYYAESVRDRFTISRDDSKNSLYLQMNSLKTEDTAMYYCVRGGYGIRGEYTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 51 - protein, 592 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSGGGGSGGGGSGGGGSEVQLVESGGGLVQPGGSLRLSCAASGFTFNTNAMNWVRQAPGKGLEWVGRIRTKSNNYATYYAESVRDRFTISRDDSKNSLYLQMNSLKTEDTAMYYCVRGGYGIRGEYTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 118 aa, synthetic construct
EVQLVQSGAEVKKPGATVKISCKVSGYSITDYLLHWVQQAPGKGLEWMGWIDPEDAKTKYGQKFQSRVTITADTSTDTAYMELSSLRSEDTAVYYCAANSGTGTMDAWGQGTLVTVSS

SEQ ID NO: 53 - protein, 107 aa, synthetic construct
DIVMTQSPATLSVSPGERATLSCKASQNVGSNVDWYQQKPGQSPRVLIYKASIRYTGIPARFSGSGSGTEFTLTISILQSEDFAVYFCMQSKSYPITFGQGTKLEIK

SEQ ID NO: 54 - protein, 330 aa, synthetic construct [sequence omitted]

SEQ ID NO: 55 - protein, 107 aa, synthetic construct [sequence omitted]

SEQ ID NO: 56 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 355 aa, synthetic construct
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 58 - protein, 461 aa, synthetic construct
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

(19) United States
(12) Patent Application Publication
(10) Pub. No.: US 2026/0055201 A1
(43) Pub. Date: Feb. 26, 2026

(54) BISPECIFIC ANTIBODY AND USE THEREOF

(71) Applicant: SHENGHE (CHINA) BIOPHARMACEUTICAL CO., LTD., Nanjing, Jiangsu (CN)

(72) Inventors: Chong ZHOU, Nanjing (CN); Jinhua ZHOU, Nanjing (CN); Chongbing WU, Nanjing (CN); Xiaoling JIANG, Nanjing (CN); Liusong YIN, Nanjing (CN); Huabing YANG, Nanjing (CN)

(73) Assignee: SHENGHE (CHINA) BIOPHARMACEUTICAL CO., LTD., Nanjing, Jiangsu (CN)

(21) Appl. No.: 19/104,799

(22) PCT Filed: Aug. 18, 2023
(86) PCT No.: PCT/CN2023/113766

§ 371 Date: Feb. 19, 2025

(30) Foreign Application Priority Data
Aug. 19, 2022 (WO) PCT/CN2022/113463

(51) Int. Cl. C07K 16/28

(52) U.S. Cl. CPC C07K 16/2878; C07K 16/2866; C07K 2317/31; C07K 2317/569; C07K 2317/732; C07K 2317/76; C07K 2317/92

(57) ABSTRACT

A bispecific antibody, including: (a) a first antibody or antigen-binding fragment thereof that specifically binds to a first antigen; and (b) a second antibody or antigen-binding fragment thereof that specifically binds to a second antigen; where the first antigen is a T cell immunomodulator, and the second antigen is TNFR2. The bispecific antibody is a T cell regulator of tumor microenvironment, which can stimulate the proliferation of CD8+T cells, activate CD8+T cells, and release anti-tumor factors, such as IFNγ and IL2 by using a TNFR2 agonistic antibody; or can block TNF-TNFR2 and inhibit the proliferation of Treg by using a TNFR2 antagonistic antibody. At the same time, it can further relieve immunosuppression, increase the number of anti-tumor cells and solve the limitation of heterogeneity of TME by cooperating with T cell immunomodulator at the target or systemic system.

### TECHNICAL FIELD

The present invention belongs to the field of biomedicine, and specifically relates to a bispecific antibody that binds to a T cell immunomodulator and TNFR2, and use thereof.

### BACKGROUND

Monoclonal antibodies (mAb) have been widely used to treat a variety of human diseases, including cancer, autoimmune diseases, infectious diseases and cardiovascular diseases. At present, there are over 80 types of monoclonal antibodies, including mouse derived, fully humanized, and chimeric antibodies, which have been approved by the FDA for therapeutic use. Most of these antibodies are monospecific antibodies that recognize a single epitope and can be selected to activate or inhibit the activity of target molecules through this single epitope. For example, trastuzumab is one of the best-selling anti-cancer protein therapies, which blocks the growth of cancer cells by attaching itself to Her2 to prevent the attachment of human epidermal growth factor to Her2. Trastuzumab can also stimulate the body's own immune cells to destroy cancer cells. However, many physiological reactions require cross-linking or conjugation of two or more different proteins or protein subunits that are yet to be triggered. Taking the activation of heteromeric cell surface receptor complexes as an example, for these receptor complexes, activation is usually achieved through the interaction of ligands with multiple structural domains on different proteins, resulting in close correlation activation of one or two receptor components.

Bispecific antibody (BsAb) refers to an antibody molecule that can simultaneously bind to two (or more) different antigenic epitopes. Compared with traditional monoclonal antibodies, bispecific antibodies have a unique mechanism of action: (1) bispecific antibodies can simultaneously bind to two or more different antigen molecules or different epitopes of the same molecule, while combination therapy often does not have this effect. (2) Mediating cell-cell interactions, bispecific antibodies can bind to two antigens on effector and target cells respectively, bridging the gap between effector and target cells and promoting cell-cell interactions, such as mediating immune cell killing of tumor cells. Therefore, bispecific antibodies have unique advantages that traditional monoclonal antibodies do not possess.

Tumor necrosis factor receptor 2 (TNFR2), also known as tumor necrosis factor receptor superfamily 1B (TNFRSFlB) and CD120b, is a 75-KDA type I transmembrane protein that belongs to the tumor necrosis factor receptor superfamily. It contains an extracellular domain (ECD, residues 1-257) and an intracellular domain (ICD, residues 288-461) with a TRAF2 binding domain. In normal T cells, the TNFA-TNFR2 interaction triggers cell survival signals through the NFKB signaling pathway. However, in autoimmune T cells, the interaction between TNFA-TNFR2 triggers apoptotic signals through the Caspase pathway.

Currently, TNFR2 has been shown to enhance the activation of effector T cells (Teff) and reduce the inhibitory effect mediated by regulatory T cells (Treg). The activation of TNFR2 induces signal transduction through the mitogen activated protein kinase (MAPK) signaling pathway, which coordinates the transcription of genes that promote evasion of cell apoptosis and proliferation through TRAF2/3 signaling and NFκB mediation. TNFR2 can be expressed not only on cancer cells and infiltrating tumor Tregs, but also on effector Teff cells. Research has shown that TNFR2 agonistic antibodies can stimulate the proliferation of CD8+T cells, activate CD8+T cells, and release anti-tumor factors such as IFNγ and IL2; TNFR2 antagonistic antibodies can block TNF-TNFR2 and inhibit Treg proliferation.

T cell co-stimulatory immunomodulators are a class of auxiliary molecules involved in immune responses. In the recognition of antigens by cells, the specific binding of T cell co-stimulatory immunomodulators can effectively enhance the adhesion between T cells and other cells, transmit antigen stimulation information, participate in the immune activation process of cells, and play an important role in cell antigen recognition and immune response.

T cell co-inhibitory immunomodulators negatively regulate immune responses. Multiple studies have shown that co-inhibitory molecules are an important strategy for tumors to evade immune surveillance. By inhibiting immune cell activity through co-inhibitory signals, they promote cancer occurrence, cell proliferation, metastasis, and further deterioration of tumors. In some cases, co-inhibitory receptors (such as CTLA-4 Ig) or monoclonal antibodies against co-inhibitory molecules in diseases can relieve immune suppression signals, restore immune cell function, and further enhance effector cell activity and quantity by combining with T cell regulators, clearing Tregs in the tumor microenvironment, increasing the recognition and killing of tumor effector T cells, etc., achieving the goal of treating tumors.

Similarly, some studies have shown that the loss and mutation of co-inhibitory molecules lead to the occurrence of autoimmune diseases in mice and humans, indicating that the downregulation signal of co-inhibitory molecules plays an important role in preventing and treating autoimmune diseases. In some cases, monoclonal antibodies against co-inhibitory receptors or excitatory anti co-inhibitory modulators can inhibit the function of self reactive T cells. The negative regulation of immune response by T cell co-inhibitory immunomodulators is an effective way to induce tolerance in the body and treat autoimmunity.

Research has shown that immunotherapy, such as the microenvironment of tumor lesions, can be successfully applied to tumor diseases. The tumor microenvironment is influenced by factors such as age, environment, and tumor type, and exhibits heterogeneity, requiring synergistic treatment through multiple mechanisms of action. T cells are an important component of anti-tumor therapy, and the synergistic use of multiple mechanisms of action can benefit cancer patients.

### SUMMARY

In the present invention, the inventor developed a bispecific antibody with excellent performance. The bispecific antibody of the present invention can stimulate the proliferation of CD8+T cells using TNFR2 agonist antibodies, can activate CD8+T cells, and release anti-tumor factors such as IFNγ and IL2; alternatively, TNFR2 antagonist antibodies can be used to block TNF-TNFR2 and inhibit Treg proliferation. At the same time, in synergy with T cell immunomodulators at the target or systemic level, further immune suppression can be relieved, the number of anti-tumor cells can be increased, and the limitations of TME heterogeneity can be resolved. The present invention combines TNFR2 agonist or antagonist antibodies with other T cell co-stimulatory immunomodulators or T cell co-inhibitory immunomodulators to form a dual antibody, thereby regulating the Teff and Treg ratio, reversing the proportion of immune cells in the tumor microenvironment, further relieving immune suppression, increasing the number of anti-tumor immune cells, and promoting tumor cell apoptosis in multiple ways.

The present invention provides a bispecific antibody, comprising: (a) a first antibody or antigen-binding fragment thereof that specifically binds to a first antigen; and (b) a second antibody or antigen-binding fragment thereof that specifically binds to a second antigen; wherein the first antigen is a T cell immunomodulator, and the second antigen is TNFR2.

In some embodiments, the T cell immunomodulator is a T cell co-stimulatory immunomodulator or a T cell co-inhibitory immunomodulator.

In some embodiments, the first antibody or antigen-binding fragment thereof comprises a heavy chain and a light chain; the second antibody or antigen-binding fragment thereof comprises scFv or VHH.

In some embodiments, the heavy chain variable region of one heavy chain and the light chain variable region of one light chain of the first antibody form an antigen-binding site, and the heavy chain variable region of another heavy chain and the light chain variable region of another light chain form an antigen-binding site.

In some embodiments, the bispecific antibody comprises one first antibody or antigen-binding fragment thereof and one or more scFv.

In some embodiments, the bispecific antibody comprises one first antibody or antigen-binding fragment thereof and one scFv, wherein the scFv is connected to the N-terminus of the heavy chain of the first antibody or antigen-binding fragment thereof.

In some embodiments, the bispecific antibody comprises one first antibody or antigen-binding fragment thereof and one scFv, wherein the scFv is connected to the C-terminus of the heavy chain of the first antibody or antigen-binding fragment thereof.

In some embodiments, the bispecific antibody comprises one first antibody or antigen-binding fragment thereof and two scFvs.

In some embodiments, the two scFvs are respectively connected to the N-terminus of two heavy chains of the first antibody or antigen-binding fragment thereof.

In some embodiments, the two scFvs are respectively connected to the C-terminus of two heavy chains of the first antibody or antigen-binding fragment thereof.

In some embodiments, the bispecific antibody comprises one first antibody that specifically binds to tumor associated antigen (TAA) and two scFvs that specifically bind to TNFR2, wherein the two scFvs that specifically bind to TNFR2 are respectively connected to the N-terminus of two heavy chains of the first antibody.

In some embodiments, the bispecific antibody comprises one first antibody that specifically binds to tumor associated antigen (TAA) and two scFvs that specifically bind to TNFR2, wherein the two scFvs that specifically bind to TNFR2 are respectively connected to the C-terminus of two heavy chains of the first antibody.

In some embodiments, the bispecific antibody comprises two first polypeptide chains and two second polypeptide chains, and for each of the polypeptide chains: (a) the first polypeptide chain independently comprises the light chain of the first antibody or antigen-binding fragment thereof; and (b) the second polypeptide chain independently comprises the heavy chain of the first antibody or antigen-binding fragment thereof and the scFv.

In some embodiments, the two first polypeptide chains of the bispecific antibody are identical or different, and/or the two second polypeptide chains are identical or different.

In some embodiments, the bispecific antibody comprises one first antibody or antigen-binding fragment thereof and one or more VHH.

In some embodiments, the bispecific antibody comprises one first antibody or antigen-binding fragment thereof and one VHH, wherein the VHH is connected to the N-terminus of the heavy chain of the first antibody or antigen-binding fragment thereof.

In some embodiments, the bispecific antibody comprises one first antibody or antigen-binding fragment thereof and one VHH, wherein the VHH is connected to the C-terminus of the heavy chain of the first antibody or antigen-binding fragment thereof.

In some embodiments, the bispecific antibody comprises one first antibody or antigen-binding fragment thereof and two VHHs.

In some embodiments, the two VHHs are respectively connected to the N-terminus of two heavy chains of the first antibody or antigen-binding fragment thereof.

In some embodiments, the two VHHs are respectively connected to the C-terminus of two heavy chains of the first antibody or antigen-binding fragment thereof.

In some embodiments, the bispecific antibody comprises one first antibody that specifically binds to tumor associated antigen (TAA) and two VHHs that specifically bind to TNFR2, which are respectively connected to the N-terminus of two heavy chains of the first antibody.

In some embodiments, the bispecific antibody comprises one first antibody that specifically binds to tumor associated antigen (TAA) and two VHHs that specifically bind to TNFR2, which are respectively connected to the C-terminus of two heavy chains of the first antibody.

In some embodiments, the bispecific antibody comprises two first polypeptide chains and two second polypeptide chains, and for each of the polypeptide chains: (a) the first polypeptide chain independently comprises the light chain of the first antibody or antigen-binding fragment thereof; and (b) the second polypeptide chain independently comprises the heavy chain of the first antibody or antigen-binding fragment thereof and the VHH.

In some embodiments, the two first polypeptide chains of the bispecific antibody are identical or different, and/or the two second polypeptide chains are identical or different.

In some embodiments, the T cell co-stimulatory immunomodulator is selected from 4-1BB, OX40, B7H2, GITR, CD48, HVEM, Nectin-2, CD40, CD30, CD28, CD27, B7-1, B7-2, LIGHT, B7H6, 2B4, CD28H or NKp30.

In some embodiments, the T cell co-inhibitory immunomodulator is selected from CCR8, PD-1, PD-L1, PD-L2, B7H3, B7H4, B7H5, BLTA, CD96, CD47, CD155, CTLA-4, LAG-3, TIGIT, TIM-3, CD111, DNAM-1, Galectin-9, Nectin-3, PVRIG, SIRP α, SIRP β, SIRP α V2 or CD160.

In some embodiments, the heavy chain variable region and light chain variable region of the scFv are connected by a linker L1.

In some embodiments, the scFv is linked to the N-terminus or C-terminus of the heavy chain of the first antibody or antigen-binding fragment thereof by a linker L2.

In some embodiments, the VHH is connected to the N-terminus or C-terminus of the heavy chain of the first antibody or antigen-binding fragment thereof by a linker L2.

In some embodiments, the linker L1 and linker L2 may be the same or different. In some embodiments, the linker L1 and/or linker L2 has an amino acid sequence as shown in (G₄S)ₓ, where x is an integer selected from 1-6; preferably, the linker L1 and/or linker L2 is (G₄S)₂, (G₄S)₃, or (G₄S)₄.

In some embodiments, the heavy chain of the first antibody or antigen-binding fragment thereof comprises a heavy chain variable region and a heavy chain constant region, and the light chain comprises a light chain variable region and a light chain constant region; preferably, the first antibody or antigen-binding fragment thereof is a full-length antibody.

In some embodiments, the heavy chain of the first antibody or antigen-binding fragment thereof comprises a first Fc region and a second Fc region. In some embodiments, the first Fc region and the second Fc region may be the same or different. In some embodiments, the first Fc region or the second Fc region is selected from IgG, IgA, IgD, IgE, IgM, or variants thereof. In some embodiments, the first Fc region or the second Fc region is selected from IgG1, IgG2, IgG3, IgG4, or variants thereof. In some embodiments, the first Fc region or the second Fc region contains one or more amino acid mutations, preferably amino acid substitutions, insertions, or deletions.

In some embodiments, the first antibody or antigen-binding fragment thereof specifically binds to 4-1BB, wherein HCDR1 of the first antibody or antigen-binding fragment thereof is as shown in SEQ ID NO: 2, or is a sequence having at least 80% identity to SEQ ID NO: 2; HCDR2 is as shown in SEQ ID NO: 3, or is a sequence having at least 80% identity to SEQ ID NO: 3; HCDR3 is as shown in SEQ ID NO: 4, or is a sequence having at least 80% identity to SEQ ID NO: 4; LCDR1 is as shown in SEQ ID NO: 6, or is a sequence having at least 80% identity to SEQ ID NO: 6; LCDR2 is as shown in SEQ ID NO: 7, or is a sequence having at least 80% identity to SEQ ID NO: 7; and LCDR3 is as shown in SEQ ID NO: 8, or is a sequence having at least 80% identity to SEQ ID NO: 8.

In some embodiments, the first antibody or antigen-binding fragment thereof specifically binds to CCR8, wherein HCDR1 of the first antibody or antigen-binding fragment thereof is as shown in SEQ ID NO: 10, or is a sequence having at least 80% identity to SEQ ID NO: 10; HCDR2 is as shown in SEQ ID NO: 11, or is a sequence having at least 80% identity to SEQ ID NO: 11; HCDR3 is as shown in SEQ ID NO: 12, or is a sequence having at least 80% identity to SEQ ID NO: 12; LCDR1 is as shown in SEQ ID NO: 14, or is a sequence having at least 80% identity to SEQ ID NO: 14; LCDR2 is as shown in SEQ ID NO: 15, or is a sequence having at least 80% identity to SEQ ID NO: 15; and LCDR3 is as shown in SEQ ID NO: 16, or is a sequence having at least 80% identity to SEQ ID NO: 16.

In some embodiments, the scFv specifically binds to TNFR2, and HCDR1 of the scFv is as shown in SEQ ID NO: 18, or is a sequence having at least 80% identity to SEQ ID NO: 18; HCDR2 is as shown in SEQ ID NO: 19, or is a sequence having at least 80% identity to SEQ ID NO: 19; HCDR3 is as shown in SEQ ID NO: 20, or is a sequence having at least 80% identity to SEQ ID NO: 20; LCDR1 is as shown in SEQ ID NO: 22, or is a sequence having at least 80% identity to SEQ ID NO: 22; LCDR2 is as shown in SEQ ID NO: 23, or is a sequence having at least 80% identity to SEQ ID NO: 23; and LCDR3 is as shown in SEQ ID NO: 24, or is a sequence having at least 80% identity to SEQ ID NO: 24.

In some embodiments, the VHH specifically binds to TNFR2, and HCDR1 of the VHH is as shown in SEQ ID NO: 26, or is a sequence having at least 80% identity to SEQ ID NO: 26; HCDR2 is as shown in SEQ ID NO: 27, or is a sequence having at least 80% identity to SEQ ID NO: 27; and HCDR3 is as shown in SEQ ID NO: 28, or is a sequence having at least 80% identity to SEQ ID NO: 28.

In some embodiments, the first antibody or antigen-binding fragment thereof specifically binds to 4-1BB, wherein the heavy chain variable region VH of the first antibody or antigen-binding fragment thereof is as shown in SEQ ID NO: 1, or is a sequence having at least 80% identity to SEQ ID NO: 1; and the light chain variable region VL is as shown in SEQ ID NO: 5, or is a sequence having at least 80% identity to SEQ ID NO: 5.

In some embodiments, the first antibody or antigen-binding fragment thereof specifically binds to CCR8, wherein the heavy chain variable region VH of the first antibody or antigen-binding fragment thereof is as shown in SEQ ID NO: 9, or is a sequence having at least 80% identity to SEQ ID NO: 9; and the light chain variable region VL is as shown in SEQ ID NO: 13, or is a sequence having at least 80% identity to SEQ ID NO: 13.

In some embodiments, the scFv specifically binds to TNFR2, and the heavy chain variable region VH of the scFv is as shown in SEQ ID NO: 17, or is a sequence having at least 80% identity to SEQ ID NO: 17; and the light chain variable region VL is as shown in SEQ ID NO: 21, or is a sequence having at least 80% identity to SEQ ID NO: 21.

In some embodiments, the VHH specifically binds to TNFR2, and the heavy chain variable region VH of the VHH is as shown in SEQ ID NO: 25, or is a sequence having at least 80% identity to SEQ ID NO: 25.

In some embodiments, the first polypeptide chain of the bispecific antibody is selected from the amino acid sequence of any one of SEQ ID NOs: 30-34, 42-46, or is an amino acid sequence having at least 80% identity to the amino acid sequence of any one of SEQ ID NOs: 30-34, 42-46; and the second polypeptide chain of the bispecific antibody is selected from the amino acid sequence of any one of SEQ ID NOs: 35-41, 47-51, or is an amino acid sequence having at least 80% identity to the amino acid sequence of any one of SEQ ID NOs: 35-41, 47-51.

In some embodiments, the bispecific antibody comprises:
(1) the first polypeptide chain as shown in SEQ ID NO: 30, and the second polypeptide chain as shown in SEQ ID NO: 35;
(2) the first polypeptide chain as shown in SEQ ID NO: 31, and the second polypeptide chain as shown in SEQ ID NO: 36;
(3) the first polypeptide chain as shown in SEQ ID NO: 30, and the second polypeptide chain as shown in SEQ ID NO: 37;
(4) the first polypeptide chain as shown in SEQ ID NO: 32, and the second polypeptide chain as shown in SEQ ID NO: 36;
(5) the first polypeptide chain as shown in SEQ ID NO: 30, and the second polypeptide chain as shown in SEQ ID NO: 38;
(6) the first polypeptide chain as shown in SEQ ID NO: 30, and the second polypeptide chain as shown in SEQ ID NO: 40;
(7) the first polypeptide chain as shown in SEQ ID NO: 33, and the second polypeptide chain as shown in SEQ ID NO: 36;
(8) the first polypeptide chain as shown in SEQ ID NO: 30, and the second polypeptide chain as shown in SEQ ID NO: 39;
(9) the first polypeptide chain as shown in SEQ ID NO: 30, and the second polypeptide chain as shown in SEQ ID NO: 41;
(10) the first polypeptide chain as shown in SEQ ID NO: 34, and the second polypeptide chain as shown in SEQ ID NO: 36;
(11) the first polypeptide chain as shown in SEQ ID NO: 42, and the second polypeptide chain as shown in SEQ ID NO: 48;
(12) the first polypeptide chain as shown in SEQ ID NO: 43, and the second polypeptide chain as shown in SEQ ID NO: 47;
(13) the first polypeptide chain as shown in SEQ ID NO: 42, and the second polypeptide chain as shown in SEQ ID NO: 49;
(14) the first polypeptide chain as shown in SEQ ID NO: 44, and the second polypeptide chain as shown in SEQ ID NO: 47;
(15) the first polypeptide chain as shown in SEQ ID NO: 42, and the second polypeptide chain as shown in SEQ ID NO: 50;
(16) the first polypeptide chain as shown in SEQ ID NO: 45, and the second polypeptide chain as shown in SEQ ID NO: 47;
(17) the first polypeptide chain as shown in SEQ ID NO: 42, and the second polypeptide chain as shown in SEQ ID NO: 51; or
(18) the first polypeptide chain as shown in SEQ ID NO: 46, and the second polypeptide chain as shown in SEQ ID NO: 47.

The present invention provides an isolated nucleic acid molecule, comprising a nucleotide sequence encoding the bispecific antibody described in any of the foregoing. Preferably, the isolated nucleic acid molecule comprises a nucleotide sequence encoding the first polypeptide chain of the bispecific antibody described in any of the foregoing. Preferably, the isolated nucleic acid molecule comprises a nucleotide sequence encoding the second polypeptide chain of the bispecific antibody described in any of the foregoing.

The present invention provides a multifunctional fusion protein, comprising the bispecific antibody described in any of the foregoing.

In some embodiments, the multifunctional fusion protein further comprises one or more third antibody or antigen-binding portion thereof that specifically binds to other antigens. In some embodiments, the antigen that binds to the third antibody or antigen-binding portion thereof is selected from tumor-associated antigen (TAA) or immune checkpoint molecule. In some embodiments, the antigen that binds to the third antibody or antigen-binding portion thereof is selected from GPC3, CD19, CD20(MS4A1), CD22, CD24, CD30, CD33, CD38, CD40, CD123, CD133, CD138, CDK4, CEA, Claudin18.2, AFP, ALK, B7H2, B7H3, B7H5, BAGE Protein, BCMA, BIRC5 (survival factor), BIRC7, β-catenin, BRC-AB1, BRCA1, BORIS, CA9, CA125, carbonic anhydrase IX, caspase-8, CALR, CCR5, NA17, NKG2D, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE protein, cyclin-B1 CYP1B1, EGFR, EGFRvIII, ErbB2/Her2, ErbB3, ErbB4, ETV6-AML, EpCAM, EphA2, Fra-1, FOLR1, GAGE Protein GD2, GD3, GloboH, GM3, gp100, Her2, HLA/B-raf, HLA/k-ras, HLA/MAGE-A3, hTERT, IL13Rα2, LMP2, κ-Light, LeY, MAGE-1, MAGE-2, MAGE-3, MAGE-4, MAGE-6, MAGE-12, MART-1, Mesothelin ML-IAP, MOv-γ, Mucd, Muc2, Muc3, Muc4, Muc5, Muc16, MUM1, Ras, RGS5, Rho, ROR1, SART-1, SART-3, STEAP1, STEAP2, TAG-72, TGF-β, TMPRSS2, Tang Nuo antigen TRP-1, TRP-2, Tyrosinase and urinary protein-3, 5T4 PD-L1, CTLA4, PD-L2, PD-1, 4-1BB, CD47, TIGIT, GITR, TIM3, ILT4, TREM2, LAG3, CD27, B7H4, CD48, HVEM, Nectin-2, CD28, B7-1, B7-2, B7H6, 2B4, CD28H, NKp30, BLTA, CD96, CD155, CTLA-4, LAG-3, TIM-3, CD111, DNAM-1, Galectin-9, Nectin-3, PVRIG, SIRP α, SIRP β, SIRP αV2 or CD160.

In some embodiments, the multifunctional fusion protein further comprises a cytokine. In some embodiments, the cytokines are selected from IL-1, IL-2, IL-2 Ra, IL-2 RP, IL-3, IL-3 Rα, IL-4, IL-4 Rα, IL-5, IL-5 Rα, IL-6, IL-6 Rα, IL-7, IL-7 Rα, IL-8, IL-9, IL-9 Rα, IL-10, IL-10R1, IL-10R2, IL-11, IL-11 Rα, IL-12, IL-12 Rα, IL-12 Rβ2, IL-12 Rβ 1, IL-13, IL-13 Rα, IL-13 Rα2, IL-14, IL-15, IL-15Rα sushi, IL-16, IL-17, IL-18, IL-19, IL-20, IL-20R1, IL-20R2, IL-21, IL-21 Rα, IL-22, IL-23, IL-23R, IL-27 R, IL-31 R, TGF, VEGF, IFNγ, IFNα or GM-CSF.

The present invention also provides use of the bispecific antibody described in any of the foregoing or the multifunctional fusion protein described in any of the foregoing in the preparation of a drug for the treatment of cancer. In some embodiments, the cancer is selected from the group consisting of human glioblastoma, human pharyngeal cancer, adrenal tumors AIDS related cancers, acinar soft tissue sarcoma, astrocytoma, bladder cancer, bone cancer, brain and spinal cord cancer, metastatic brain tumor, breast cancer, carotid body tumor, cervical cancer, chondrosarcoma, chordoma, renal chromophobe cell cancer, clear cell cancer, colon cancer, colorectal cancer, connective tissue proliferative small round cell tumor, ependymal cell tumor, Ewing tumor, extraskeletal myxoid chondrosarcoma, osteofibrous dysplasia, fibrous dysplasia of bone, gallbladder or bile duct cancer, gastric cancer, gestational trophoblastic disease, germ cell tumor, head and neck cancer, hepatocellular carcinoma, pancreatic islet cell tumor, Kaposi's sarcoma, renal cancer, leukemia, liposarcoma/malignant lipomatous tumor, liver cancer, lymphoma, lung cancer, neuroblastoma, melanoma, meningioma, multiple endocrine neoplasia, multiple myeloma Myelodysplastic syndrome, neuroblastoma, neuroendocrine tumor, ovarian cancer, pancreatic cancer, papillary thyroid cancer, parathyroid adenoma, pediatric cancer, peripheral neurilemmoma, pheochromocytoma, pituitary tumor, prostate cancer, posterior uveal melanoma, renal metastatic cancer, rhabdomyoid tumor, rhabdomyosarcoma, sarcoma, skin cancer, soft tissue sarcoma, squamous cell carcinoma, synovial sarcoma, testicular cancer, thymic cancer, thymoma, thyroid metastatic cancer or uterine cancer.

The present invention also provides use of the bispecific antibody described in any of the foregoing or the multifunctional fusion protein described in any of the foregoing for the preparation of a drug for treating autoimmune diseases. In some embodiments, the autoimmune disease is selected from graft-versus-host disease, rheumatoid arthritis, Crohn's disease, multiple sclerosis, colitis, psoriasis, autoimmune uveitis, pemphigus, epidermolysis bullosa or type I diabetes.

In some embodiments, the use is achieved by one or more of tumor immunotherapy, cell therapy, or gene therapy.

The present invention also provides a pharmaceutical composition, comprising the bispecific antibody described in any of the foregoing and a pharmaceutically acceptable carrier, diluent, or excipient.

The present invention also provides a pharmaceutical composition, comprising the multifunctional fusion protein described in any of the foregoing and a pharmaceutically acceptable carrier, diluent, or excipient.

The present invention also provides an antibody-drug conjugate, comprising the bispecific antibody described in any of the foregoing.

In some embodiments, the conjugate drug is selected from a cytotoxic agent, a small molecule chemical agent, or an immunotoxin.

### Abbreviations and Definitions of Terms

Use the following abbreviations in this present invention. VH: antibody heavy chain variable region; VL: antibody light chain variable region; CDR: the complementary determining region in the immunoglobulin variable region; IgG: immunoglobulin G.

The term “antibody” refers to natural immunoglobulin or immunoglobulin prepared through partial or complete synthesis. Antibodies can be reconstructed and isolated from natural resources such as plasma or serum, or from the culture supernatant of hybridoma cells that produce antibodies, animal immune serum, and phage library screening. Alternatively, partial or complete synthesis can be achieved through techniques such as genetic recombination. Preferred antibodies include, for example, antibodies of isotypes of immunoglobulin or subclasses of these isotypes. It is known that human immunoglobulin includes nine categories (isotypes): IgG1, IgG2, IgG3, IgG4, IgAQ1, IgA2, IgD, IgE, and IgM. Among these isotypes, the antibodies of the present invention may include IgG1, IgG2, IgG3, and/or IgG4.

The antibodies of the present invention are immunoglobulin molecules composed of two pairs of polypeptide chains, each pair having one light chain (LC) and one heavy chain (HC). Each heavy chain is composed of a heavy chain variable region (VH) and a heavy chain constant region (CH). The heavy chain constant region consists of three structural domains (CH1, CH2, and CH3). Each light chain consists of a light chain variable region (VL) and a light chain constant region (CL), or only a light chain constant region (CL). The constant region of the light chain is composed of a structural domain CL. The constant domain does not directly participate in the binding of antibodies to antigens, but exhibits multiple effector functions, such as mediating the binding of immunoglobulins to host tissues or factors, including various cells of the immune system (e.g. effector cells) and the first component of the classical complement system (C1q). The VH and VL regions can also be subdivided into regions with high variability, known as complementarity determining regions (CDRs), interspersed with more conservative regions called framework regions (FRs). Each VH and VL is composed of three CDRs and four FRs arranged in the following order: FR1, CDR1, FR2, CDR2, FR3, CDR3, and FR4 from the amino terminus to the carboxyl terminus. The variable regions (VH and VL) of each heavy/light chain pair form antigen-binding sites.

The term “bispecific antibody” refers to a protein molecule that can specifically bind to two target antigens or target antigen epitopes. In the present invention, “bispecific antigen-binding proteins” containing antibodies or antigen-binding fragments (such as Fab, scFv, etc.) can be used interchangeably with “bispecific antibodies”.

The term “antigen-binding fragment” of an antibody refers to a peptide fragment of the antibody, such as a peptide fragment of a full-length antibody, that maintains the ability to specifically bind to the same antigen bound by the full-length antibody and/or compete with the full-length antibody for specific binding to the antigen, also known as the “antigen-binding portion”. Antibody antigen-binding fragments can be produced through recombinant DNA technology or through enzymatic or chemical cleavage of intact antibodies. Non limiting examples of antigen-binding fragments include Fab, Fab′, F(ab′)2, Fd, Fv, dAb, and complementarity determining region (CDR) fragments, single chain antibodies (e.g. scFv), chimeric antibodies, diabodies, linear antibodies, nanobodies (e.g. technology from Ablinx), domain antibodies (e.g. technology from Domantis), and peptides containing at least a portion of antibodies sufficient to confer specific antigen-binding ability to the peptide.

The term “T cell immunomodulatory agent” refers to a T cell regulatory molecule that regulates the T cell response of the human immune system, monitors the killing of tumor cells, foreign pathogens, or avoids excessive activation of the immune system on healthy cells. T cell immunomodulators include T cell co-stimulatory immunomodulators and T cell co-inhibitory immunomodulators. The release of immune system co inhibition and enhancement of co stimulation can enhance anti-tumor immune response.

The term “antibody-drug conjugate” or “ADC” refers to a binding protein (such as an antibody or antigen-binding fragment thereof) that is linked to one or more conjugate drugs (which may optionally be therapeutic or cytotoxic agents). Its structure typically consists of three parts: an antibody or antibody ligand, a drug moiety, and a linker that couples the antibody or antibody ligand with the drug. ADC typically contains 1, 2, 3, 4, 5, 6, 7, 8, 9, or 10 drugs conjugated with antibodies.

The term “polypeptide” refers to any length of amino acid chain, regardless of modifications such as phosphorylation or glycosylation. The term polypeptide includes proteins and their fragments. Polypeptides can be “exogenous”, meaning they are “heterologous”, meaning they are derived from host cells, such as human peptides produced by bacterial cells. This present invention discloses polypeptides as amino acid residue sequences. Those sequences are written from left to right in the direction from the amino terminus to the carboxyl terminus. According to standard nomenclature, amino acid residue sequences are named with three letter or single letter codes.

The term “scFv” refers to a molecule that contains an antibody heavy chain variable domain (VH) and an antibody light chain variable domain (VL) connected by a linker. Such scFv molecules can have a general structure: NH2-VL-linker-VH-COOH or NH2-VH-linker-VL-COOH. Suitable existing technology linkers consist of repeated GGGGS amino acid sequences, or variants thereof, such as using 1-6 repeated GGGGS amino acid sequences, or variants thereof.

The term “VHH” refers to a single antigen-binding polypeptide that contains only one heavy chain variable region (VHH), derived from the variable domain of naturally heavy chain molecules without light chains, to distinguish it from the conventional VH of four chain immunoglobulins. This VHH molecule can originate from antibodies produced in camelid species such as camels, American camelids, llamas, dromedary camels, alpacas, and alpacas. Other species outside the camel family can produce heavy chain molecules that naturally lack light chains, and such VHH is within the scope of the present invention.

The term “host cell” refers to a cell that has been or can be transformed with a nucleic acid sequence to express the selected target gene. This term includes the offspring of parent cells, regardless of whether the offspring are morphologically or genetically identical to the original parent cells, as long as the offspring contain the selected target gene. Commonly used host cells include bacteria, yeast, mammalian cells, etc.

The term “carrier” refers to a nucleic acid molecule capable of proliferating another nucleic acid connected to it. This term includes vectors that serve as self replicating nucleic acid structures and vectors that are incorporated into the genome of host cells receiving their introduction. Some vectors can guide the expression of nucleic acids that can be connected to them, which are referred to as “expression vectors” in the present invention.

The term “pharmaceutically acceptable carrier” includes any standard drug carrier, such as phosphate buffered saline solutions, water and lotion, and various types of wetting agents.

The term “identity” is defined as the percentage of amino acid residues in the candidate sequence that are identical to those in the control peptide sequence, after aligning the sequence and introducing gaps as necessary to obtain the maximum percentage of sequence identity. The comparison for the purpose of determining percentage amino acid sequence identity can be carried out in various ways within the technical scope of this field, such as using publicly available computer software such as BLAST software or FASTA package.

The term “at least 80% identity” refers to the percentage of amino acid residues in the candidate sequence that are identical to those in the control peptide sequence being 80% or more, including 80%, 81%, 82%, 83%, 84%, 85%, 86%, 87%, 88%, 89%, 90%, 91%, 92%, 93%, 94%, 95%, 96%, 97%, 98%, 99%, and 100%.

The term “specificity” means that one of the molecules involved in specific binding does not exhibit any significant binding to a molecule or molecules different from one or more of the binding partner molecules. In addition, the term is also used when a domain containing an antibody variable region has specificity for a specific epitope among multiple epitopes in the antigen. When the epitope bound to the domain containing the antibody variable region is contained in several different antigens, the antigen-binding molecule containing the domain containing the antibody variable region can bind to various antigens with the epitope.

The term “tumor associated antigen” or “TAA” refers to a molecule (typically a protein, carbohydrate, lipid, or some combination thereof) that is fully or partially expressed on the surface of cancer cells and can be used to preferentially target pharmacological agents to cancer cells. Non limiting examples of tumor associated antigens include GPC3, CD19, CD20(MS4A1), CD22, CD24, CD30, CD33, CD38, CD40, CD123, CD133, CD138, CDK4, CEA, Claudin18.2, AFP, ALK, BAGE Protein, BCMA, BIRC5 (survival factor), BIRC7, β-catenin, BRC-AB1, BRCA1, BORIS, CA9, CA125, carbonic anhydrase IX, caspase-8, CALR, CCR5, NA17, NKG2D, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE protein, cyclin-B1 CYP1B1, EGFR, EGFRvIII, ErbB2/Her2, ErbB3, ErbB4, ETV6-AML, EpCAM, EphA2, Fra-1, FOLR1, GAGE Protein GD2, GD3, GloboH, GM3, gp100, Her2, HLA/B-raf, HLA/k-ras, HLA/MAGE-A3, hTERT, IL13Rα2, LMP2, κ-Light, LeY, MAGE-1, MAGE-2, MAGE-3, MAGE-4, MAGE-6, MAGE-12, MART-1, Mesothelin ML-IAP, MOv-γ, Mucd, Muc2, Muc3, Muc4, Muc5, Muc16, MUM1, Ras, RGS5, Rho, ROR1, SART-1, SART-3, STEAP1, STEAP2, TAG-72, TGF-β, TMPRSS2, Tang Nuo antigen TRP-1, TRP-2, Tyrosinase and urinary protein-3, 5T4, PD-L1, CTLA4, PD-L2, PD-1, 4-1BB, CD47, TIGIT, GITR, TIM3, ILT4, TREM2, LAG3, CD27 or B7H4.

The term “epitope” refers to the antigenic determinant in an antigen, and refers to the antigenic site to which the structural domain of the antigen-binding molecule containing the antibody variable region disclosed in this specification binds. Therefore, tables can be defined based on their structure. In addition, the epitope can also be defined based on the antigen-binding activity of the antigen-binding molecule that recognizes the epitope. When the antigen is a peptide or polypeptide, the epitope can be specified by the amino acid residues that form the epitope; when the epitope is a sugar chain, it can be determined by its specific sugar chain structure.

The term “positive control” refers to natural or engineered cells or antibodies that can bind to or express target proteins. The positive control in the present invention refers to a single target positive control.

The term “negative control” refers to the use of the same species source, subtype, dose, immunoglobulin, subtype, and label as the experimental sample in the same experiment, to eliminate the experimental background influence of non-specific binding samples on the experimental values, as a more explanatory control for the experimental effect.

The terms “Treg”, “Treg cells”, or “regulatory T cells” are sometimes referred to as suppressive T cells, characterized by the expression of biomarkers CD4, FOXP3, and CD25, representing a subset of T cells that regulate the immune system, maintain tolerance to self antigens, and prevent autoimmune diseases. Tregs are immunosuppressive and typically inhibit or downregulate the induction and proliferation of effector T (Teff) cells. Tregs can develop in the thymus (so-called CD4+Foxp3+“natural” Tregs) or differentiate from peripheral immature CD4+T cells, for example, after exposure to TGFβ or retinoic acid.

The terms “Teff”, “Teff cells”, or “effector T cells” are cells formed by the proliferation and differentiation of T cells after receiving antigen stimulation. Effect T cells have the function of releasing lymphokines, during which a small portion of T cells become memory T cells. When effector T cells come into contact with target cells and trigger granule exocytosis, the released perforin forms small pores on the surface of the target cells through polymerization, thereby mediating the killing effect. The process of target cell death is similar to apoptosis. At the same time, effector T cells can also release immune active substances-lymphokines, such as interleukins, interferons, etc.

There are various methods/systems in this field to define and describe CDR, which have been developed and refined for many years, including Kabat, Chothia, IMGT, AbM, and Contact. Kabat is the most commonly used definition of CDR based on sequence variability; Chothia defines CDRs based on the position of structural cyclic regions and sequence variability; the IMGT system defines CDRs based on sequence variability and position within the variable domain structure; AbM is defined based on Oxford Molecular's AbM antibody modeling software, which is a compromise between Kabat and Chothia; Contact defines CDR based on the analysis of complex crystal structures, which is similar to Chothia in multiple aspects. The CDR in the present invention is mainly divided using Kabat, and some CDR are divided using defined standards, such as HCDR1 shown in SEQ ID NO: 2, 10, 18, and 26. The division starts from the fourth position after the first cysteine in the heavy chain variable region, and HCDR1 is generally 10-12 in length, ending at the amino acid before tryptophan.

### BRIEF DESCRIPTION OF THE DRAWINGS

FIG. 1 shows an exemplary bispecific antibody comprising a full-length antibody capable of specifically recognizing T cell immunomodulators and an scFv capable of specifically recognizing TNFR2, which is connected to the C-terminus of two heavy chains of the full-length antibody by a linker.

FIG. 2 shows an exemplary bispecific antibody comprising a full-length antibody capable of specifically recognizing T cell immunomodulators and an scFv capable of specifically recognizing TNFR2, which is connected to the N-terminus of two heavy chains of the full-length antibody by a linker.

FIG. 3 shows an exemplary bispecific antibody comprising a full-length antibody capable of specifically recognizing T cell immunomodulators and a VHH capable of specifically recognizing TNFR2, which is connected to the C-terminus of two heavy chains of the full-length antibody by a linker.

FIG. 4 shows an exemplary bispecific antibody comprising a full-length antibody capable of specifically recognizing T cell immunomodulators and a VHH capable of specifically recognizing TNFR2, which is connected to the N-terminus of two heavy chains of the full-length antibody by a linker.

FIG. 5 shows an exemplary bispecific antibody comprising a full-length antibody capable of specifically recognizing T cell immunomodulators and an scFv capable of specifically recognizing TNFR2, which is connected to the C-terminus of two light chains of the full-length antibody by a linker.

FIG. 6 shows an exemplary bispecific antibody comprising a full-length antibody capable of specifically recognizing T cell immunomodulators and an scFv capable of specifically recognizing TNFR2, which is connected to the N-terminus of two light chains of the full-length antibody by a linker.

FIG. 7 shows an exemplary bispecific antibody comprising a full-length antibody capable of specifically recognizing T cell immunomodulators and a VHH capable of specifically recognizing TNFR2, which is connected to the C-terminus of two light chains of the full-length antibody by a linker.

FIG. 8 shows an exemplary bispecific antibody comprising a full-length antibody capable of specifically recognizing T cell immunomodulators and a VHH capable of specifically recognizing TNFR2, which is connected to the N-terminus of two light chains of the full-length antibody by a linker.

FIG. 9 shows the binding activity of antibodies TB-A, TB-E, TB-B and TB-F to TNFR2 protein.

FIG. 10 shows the binding activity of antibodies TB-C1 and TB-G to TNFR2 protein.

FIG. 11 shows the binding activity of antibodies TB-D1 and TB-H to TNFR2 protein.

FIG. 12 shows the binding activity of antibodies TB-C2 and TB-D2 to TNFR2 protein.

FIG. 13 shows the binding activity of antibodies TB-A, TB-E, TB-B and TB-F to 4-1BB protein.

FIG. 14 shows the binding activity of antibodies TB-C1, TB-G, TB-D1 and TB-H to 4-1BB protein.

FIG. 15 shows the binding activity of antibody TB-C2 to 4-1BB protein.

FIG. 16 shows the dual-terminal binding activities of antibodies TB-A, TB-E, TB-B and TB-F.

FIG. 17 shows the dual-terminal binding activities of antibodies TB-C1, TB-G, TB-D1 and TB-H.

FIG. 18 shows the dual-terminal binding activities of antibodies TB-A, TB-E, TB-B and TB-F.

FIG. 19 shows the dual-terminal binding activities of antibodies TB-C1, TB-G, TB-D1 and TB-H.

FIG. 20 shows the activation effect of antibodies TB-C1 and TB-D1 on HEK-293/NFκB Luci/4-1BB cells.

FIG. 21 shows the anti-tumor growth curves of antibodies TB-C2 and TB-D2.

FIG. 22 shows the ADCC activity of antibodies TB-C1 and TB-D1.

FIG. 23 shows the binding activity of antibodies TR-A to TR-H with CCR8-CHO-K1 cells.

FIG. 24 shows the binding activity of antibodies TR-A to TR-H with TNFR2-CHO-K1 cells.

FIG. 25 shows the ADCC activity of antibodies TR-A, TR-E, TR-B and TR-F.

FIG. 26 shows the ADCC activity of antibodies TR-C, TR-G, TR-D and TR-H.

FIG. 27 shows the blocking activity of antibodies TR-C, TR-G and TR-D on TNFR2-TNF binding.

FIG. 28 shows the blocking activity of antibody TR-H on TNFR2-TNF binding.

FIG. 29 shows the activation experiment results of antibodies TR-A, TR-E, TR-B and TR-F activating CD8+T cells to release IFN-7.

### DETAILED DESCRIPTION

The present invention will be further described in conjunction with the accompanying drawings and specific embodiments, and the protected content of the present invention is not limited to the following embodiments. It should also be understood that the terms used in the embodiments of the present invention are intended to describe specific embodiments, rather than to limit the scope of protection of the present invention. All variations and advantages that can be conceived by those skilled in the art without departing from the spirit and scope of the inventive concept are included in the present invention, and the appended claims and any equivalents thereof are within the scope of protection of the present invention.

### Example 1 Preparation of Bispecific Antibodies with Different Structures

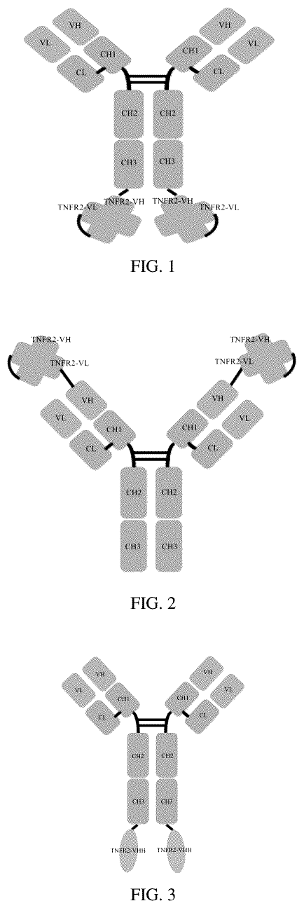

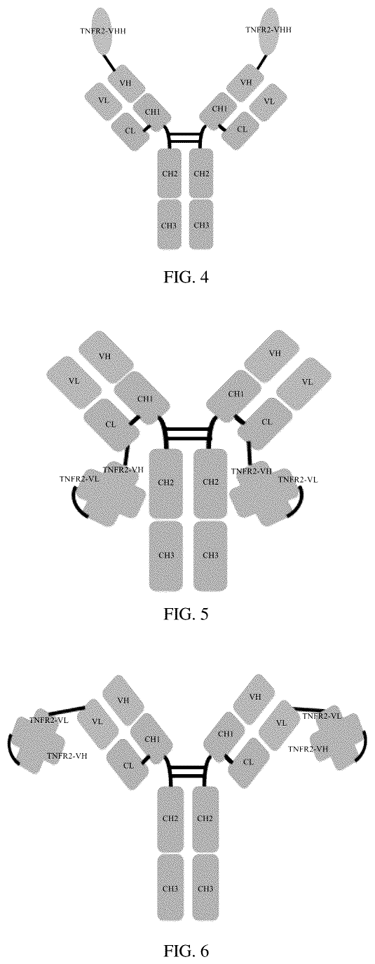

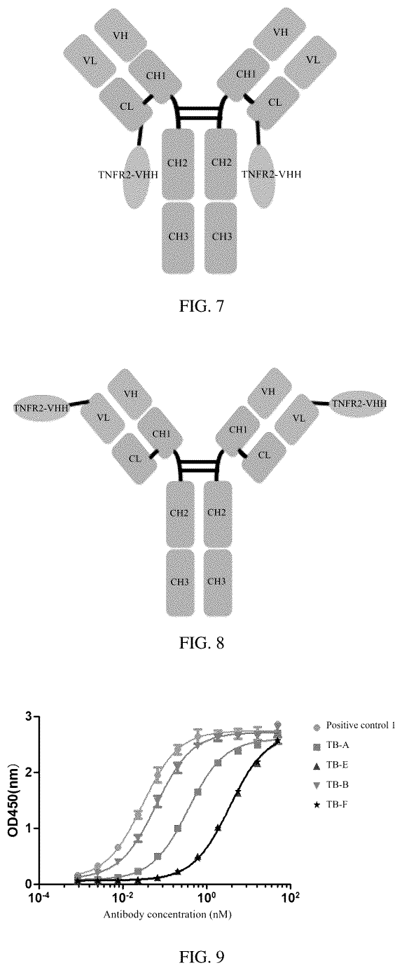

For TNFR2 and 4-1BB, bispecific antibodies were constructed according to the eight structures in FIGS. 1-8, respectively, and named TB-A to TB-H in order. For TNFR2 and CCR8, bispecific antibodies were constructed according to the eight structures in FIGS. 1-8, and named TR-A to TR-H in order. The bispecific antibodies with part of structures of the present patent simultaneously construct IgG1 and IgG4 subtypes, and the antibody name followed by “1” is the IgG1 subtype, and the antibody name followed by “2” is the IgG4 subtype, for example: antibody TB-C1 was an IgG1 subtype antibody against TNFR2 and 4-1BB targets, constructed according to the structure in FIG. 3; and antibody TB-C2 was an IgG4 subtype antibody against TNFR2 and 4-1BB targets, constructed according to the structure in FIG. 3.

The sequences of anti-4-1BB antibody, anti-CCR8 antibody as full-length antibodies, and the sequences of TNFR2 antibody as scFv and VHH are shown in Table 1.

TABLE 1
Sequence list

| | 4-1BB | CCR8 | | |
|---|---|---|---|---|
| | (full-length | (full-length | TNFR2 | TNFR2 |
| Area | antibody) | antibodies) | (scFv) | (VHH) |
| Heavy chain variable | SEQ ID NO: 1 | SEQ ID NO: 9 | SEQ ID NO: 17 | SEQ ID NO: 25 |
| region (VH) | | | | |
| HCDR1 | SEQ ID NO: 2 | SEQ ID NO: 10 | SEQ ID NO: 18 | SEQ ID NO: 26 |
| HCDR2 | SEQ ID NO: 3 | SEQ ID NO: 11 | SEQ ID NO: 19 | SEQ ID NO: 27 |
| HCDR3 | SEQ ID NO: 4 | SEQ ID NO: 12 | SEQ ID NO: 20 | SEQ ID NO: 28 |
| Light chain variable | SEQ ID NO: 5 | SEQ ID NO: 13 | SEQ ID NO: 21 | / |
| region (VL) | | | | |
| LCDR1 | SEQ ID NO: 6 | SEQ ID NO: 14 | SEQ ID NO: 22 | / |
| LCDR2 | SEQ ID NO: 7 | SEQ ID NO: 15 | SEQ ID NO: 23 | / |
| LCDR3 | SEQ ID NO: 8 | SEQ ID NO: 16 | SEQ ID NO: 24 | / |

TNFR2 antibody, anti-4-1BB antibody, and anti-CCR8 antibody were linked in a certain order through linkers to form peptide chains as shown in Table 2 and Table 3. In this embodiment, the linker used in the bispecific antibody consists of 2 GGGGS repeats (i.e. GGGGSGGGGS, abbrebyted as (G₄S)₂) or 3 GGGGS repeats (i.e. GGGGSGGSGGGGS, abbrebyted as (G₄S)₃), and the CL used in this embodiment was kappa (x) type, as shown in the sequence of SEQ ID NO: 29.

TABLE 2
Amino acid sequence of peptide chain of bispecific antibody (TNFR2+4-1BB)

| Peptide | | Amino Acid | |
|---|---|---|---|
| chain | Structural order | Sequence | Notes |
| L1 | VL(4-1BB)-CL(kappa) | SEQ ID NO: 30 | / |
| L2 | VL(4-1BB)-CL(kappa)-(G₄S)₂-VH(TNFR2)-(G₄S)₃- | SEQ ID NO: 31 | / |
| | VL(TNFR2) | | |
| L3 | VH(TNFR2)-(G₄S)₃-VL(TNFR₂)-(G₄S)₃-VL | SEQ ID NO: 32 | / |
| | (4-1BB)-CL(kappa) | | |
| L4 | VL(4-1BB)-CL(kappa)-(G₄S)₂-VHH(TNFR2) | SEQ ID NO: 33 | / |
| L5 | VHH(TNFR2)-(G₄S)₃-VL(4-1BB)-CL(kappa) | SEQ ID NO: 34 | / |
| H1 | VH(4-1BB)-CH1-CH2-CH3-(G₄S)₃-VH(TNFR2)- | SEQ ID NO: 35 | IgG1 subtype |
| | (G₄S)₃-VL(TNFR2) | | |
| H2 | VH(4-1BB)-CH1-CH2-CH3 | SEQ ID NO: 36 | IgG1 subtype |
| H3 | VH(TNFR2)-(G₄S)₃-VL(TNFR2)-(G₄S)₃-VH | SEQ ID NO: 37 | IgG1 subtype |
| | (4-1BB)-CH1-CH2-CH3 | | |
| H4 | VH(4-1BB)-CH1-CH2-CH3-(G₄S)₃-VHH(TNFR2) | SEQ ID NO: 38 | IgG1 subtype |
| H5 | VHH(TNFR2)-(G₄S)₃-VH(4-1BB)-CH1-CH2-CH3 | SEQ ID NO: 39 | IgG1 subtype |
| H6 | VH(4-1BB)-CH1-CH2-CH3-(G₄S)₃-VHH(TNFR2) | SEQ ID NO: 40 | IgG4 subtype |
| H7 | VHH(TNFR2)-(G₄S)₃-VH(4-1BB)-CH1-CH2-CH3 | SEQ ID NO: 41 | IgG4 subtype |

TABLE 3
Amino acid sequence of peptide chain of bispecific antibody (TNFR2 + CCR8)

| Peptide | | Amino Acid |
|---|---|---|
| chain | Structural order | Sequence |
| L1 | VL(CCR8)-CL(kappa) | SEQ ID NO: 42 |
| L2 | VL(CCR8)-CL(kappa)-(G₄S)₂-VH(TNFR2)-(G₄S)₃- | SEQ ID NO: 43 |
| | VL(TNFR2) | |
| L3 | VH(TNFR2)-(G₄S)₃-VL(TNFR2)-(G₄S)₃-VL(CCR8)- | SEQ ID NO: 44 |
| | CL(kappa) | |
| L4 | VL(CCR8)-CL(kappa)-(G₄S)₂-VHH(TNFR2) | SEQ ID NO: 45 |
| L5 | VHH(TNFR2)-(G₄S)₃-VL(CCR8)-CL(kappa) | SEQ ID NO: 46 |
| H1 | VH(CCR8)-CH1-CH2-CH3 | SEQ ID NO: 47 |
| H2 | VH(CCR8)-CH1-CH2-CH3-(G₄S)₃-VH(TNFR2)-(G₄S)₃- | SEQ ID NO: 48 |
| | VL(TNFR2) | |
| H3 | VH(TNFR2)-(G₄S)₃-VL(TNFR2)-(G₄S)₃-VH(CCR8)-CH1- | SEQ ID NO: 49 |
| | CH2-CH3 | |
| H4 | VH(CCR8)-CH1-CH2-CH3-(G₄S)₃-VHH(TNFR2) | SEQ ID NO: 50 |
| H5 | VHH(TNFR2)-(G₄S)₃-VH(CCR8)-CH1-CH2-CH3 | SEQ ID NO: 51 |

Based on the peptide chain combinations in Table 2 and Table 3, designed the amino acid sequence of the bispecific antibodies shown in Table 4.

TABLE 4
Amino acid sequences of bispecific antibodies

| | | The amino acid sequence |
|---|---|---|
| | The amino acid sequence | of the second peptide |
| Antibodies | of the first peptide chain | chain |
| TB-A | SEQ ID NO: 30 | SEQ ID NO: 35 |
| TB-E | SEQ ID NO: 31 | SEQ ID NO: 36 |
| TB-B | SEQ ID NO: 30 | SEQ ID NO: 37 |
| TB-F | SEQ ID NO: 32 | SEQ ID NO: 36 |
| TB-C1 | SEQ ID NO: 30 | SEQ ID NO: 38 |
| TB-C2 | SEQ ID NO: 30 | SEQ ID NO: 40 |
| TB-G | SEQ ID NO: 33 | SEQ ID NO: 36 |
| TB-D1 | SEQ ID NO: 30 | SEQ ID NO: 39 |
| TB-D2 | SEQ ID NO: 30 | SEQ ID NO: 41 |
| TB-H | SEQ ID NO: 34 | SEQ ID NO: 36 |
| TR-A | SEQ ID NO: 42 | SEQ ID NO: 48 |
| TR-E | SEQ ID NO: 43 | SEQ ID NO: 47 |
| TR-B | SEQ ID NO: 42 | SEQ ID NO: 49 |
| TR-F | SEQ ID NO: 44 | SEQ ID NO: 47 |
| TR-C | SEQ ID NO: 42 | SEQ ID NO: 50 |
| TR-G | SEQ ID NO: 45 | SEQ ID NO: 47 |
| TR-D | SEQ ID NO: 42 | SEQ ID NO: 51 |
| TR-H | SEQ ID NO: 46 | SEQ ID NO: 47 |

Gene synthesis was carried out on the various chains of bispecific antibodies with different structures designed above. Using molecular cloning technology, antibody fragments were inserted into the PCDNA3.1 vector to construct mammalian cell expression plasmids. Liposome transfection was used to introduce the host cell line CHO cells, and the fermentation supernatant was obtained using cell Fed-batch. The fermentation supernatant was purified through a series of steps such as affinity chromatography and ion exchange chromatography to obtain the constructed antibodies. Detected the expression level, purity, SDS-PAGE, etc. of the purified antibodies to confirm the characterization of the bispecific antibodies.

### Example 2 Detection of the Binding Activity of Antibodies to TNFR2 Protein by ELISA (TNFR2+4-1BB)

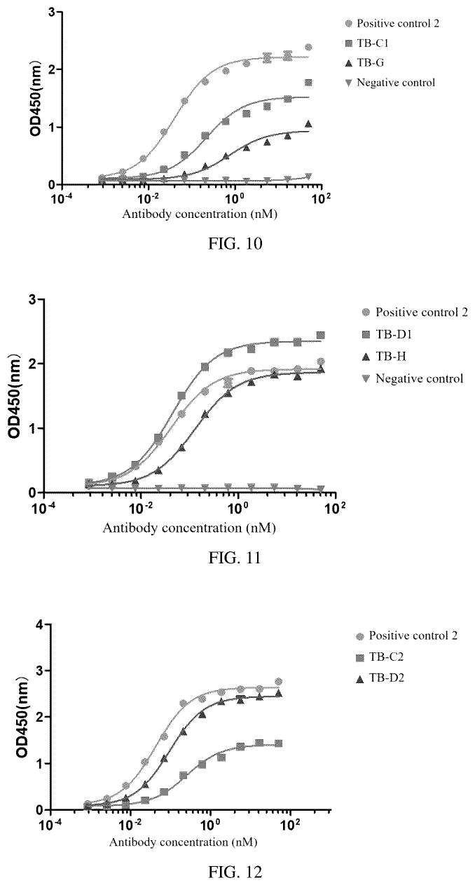

Human TNFR2 His (manufacturer: SINO, CAT: 10417-H08H, LOT: LC15NO0412) was diluted to 0.1 μg/mL with a coating solution (1×PBS, pH 7.4), added into a 96-well enzyme-linked immunosorbent assay plate at 100 μL/well, and coated overnight at 4° C. The coating solution was poured off. Each well was washed with 1×PBST at 300 μL, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. Negative control and positive controls were set up, the negative control was commercially available injectable trastuzumab (Herceptin), and the antibody sequence variable region of positive control 1 consisting of SEQ ID NO: 52 and SEQ ID NO: 53, with the addition of the constant region of human IgG1 (SEQ ID NO: 54 and SEQ ID NO: 55); the antibody sequence variable region of positive control 2 consisting of SEQ ID NO: 25. The positive control and antibody were diluted to 10 μg/mL with 3% skim milk powder as the starting concentration, and then diluted by 3-fold gradient (11 gradients in total). A blank well was set up and only the diluent was added. It was incubated at 37° C. for 1 h at 100 μL/well. The liquid in the well was discarded, washed 4 times with a plate washer, and patted dry on a flat paper. Goat anti human IgG Fc was diluted with 3% skim milk powder at 1:10000, and incubated at 37° C. for 1 h at 100 μL/well. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader. The ELISA results of antibody molecules are shown in FIGS. 9-12, indicating that antibodies TB-A to TB-H can bind to human TNFR2 protein.

### Example 3 Detection of the Binding Activity of Antibodies to 4-1BB Protein by ELISA (TNFR2+4-1BB)

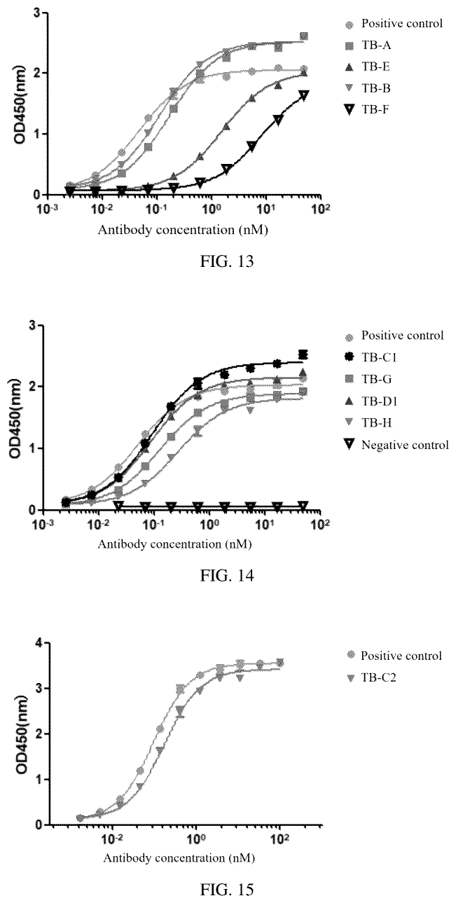

Human 4-1BB His (manufacturer: Acro, CAT: 41B-H5258, LOT: 198-2146F1-W6) was diluted to 0.2 μg/mL with a coating solution (1×PBS, pH 7.4), added into a 96-well enzyme-linked immunosorbent assay plate at 100 μL/well, and coated overnight at 4° C. The coating solution was poured off. Each well was washed with 1×PBST at 300 μL, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. Negative control and positive control were set up. The negative control was commercially available injectable trastuzumab (Herceptin). The variable region of the antibody sequence of positive control consists of SEQ ID NO: 1 and SEQ ID NO: 5, with the addition of the constant region of human IgG1 (SEQ ID NO: 54 and SEQ ID NO: 55). The positive control and antibody were diluted to 10 μg/mL with 3% skim milk powder as the starting concentration, and then diluted by 3-fold gradient (11 gradients in total), and a blank well was set up and only the diluent was added. It was incubated at 37° C. for 1 h at 100 μL/well. The liquid in the well was discarded, washed 4 times with a plate washer, and patted dry on a flat paper. Goat anti human IgG was diluted with 3% skim milk powder at 1:20000, and incubated at 37° C. for 1 h at 100 μL/well. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader. The ELISA results of antibody molecules are shown in FIGS. 13-15, indicating that antibodies TB-A to TB-H can bind to human 4-1BB protein.

### Example 4 Binding Activity at Both Ends of the Constructed Antibodies (TNFR2+4-1BB)

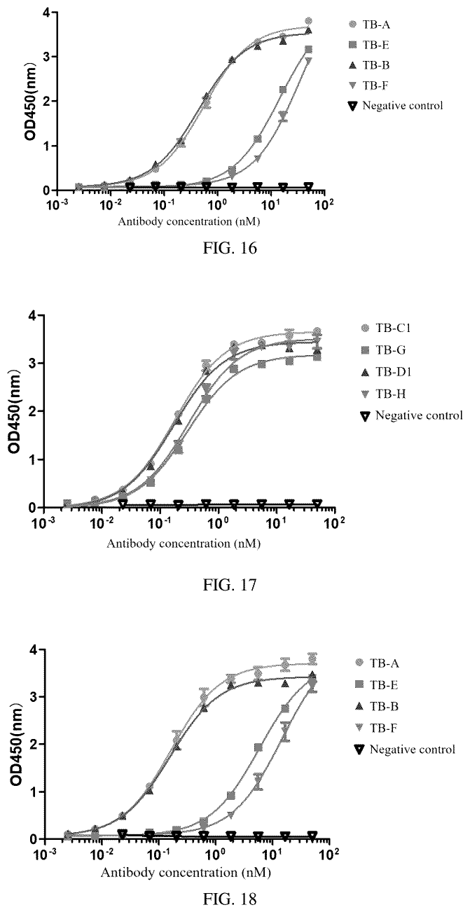

Human 4-1BB Fc (manufacturer: Acro, CAT: 41B-H5258, LOT: 198-2146F1-W6) was diluted to 0.3 μg/mL with a coating solution (1×PBS, pH 7.4) and coated into a 96-well ELISA plate at 100 μL/well at 4° C. overnight. The coating solution was poured off. Each well was washed with 1×PBST at 300 μL, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. A negative control was set up, the negative control was commercially available injectable trastuzumab (Herceptin). Starting at 50 nM, which was used as the initial concentration for 3-fold dilution, a total of 10 gradients were diluted, and a blank well was set up and only the diluent was added. It was incubated at 37° C. for 1 h at 100 μL/well. The liquid in the well was discarded, washed 4 times with a plate washer, and patted dry on a flat paper. Human TNFR2 His (manufacturer: Sino, CAT: 10417-H08H, LOT: LC15NO0412) was diluted to 0.3 μg/mL, 100 μL was added to each well, and incubated at room temperature for 1 h. The plate was washed 3 times with PBST, and then the HRP labeled his antibody was diluted with sample diluent at 1:5000, 100 μL was added to each well, and incubated at room temperature for 1 h. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader. The ELISA results of antibody molecules are shown in FIG. 16 and FIG. 17 respectively, indicating that antibodies TB-A to TB-H have binding activity at both ends.

### Example 5 Binding Activity at Both Ends of the Constructed Antibodies (TNFR2+4-1BB)

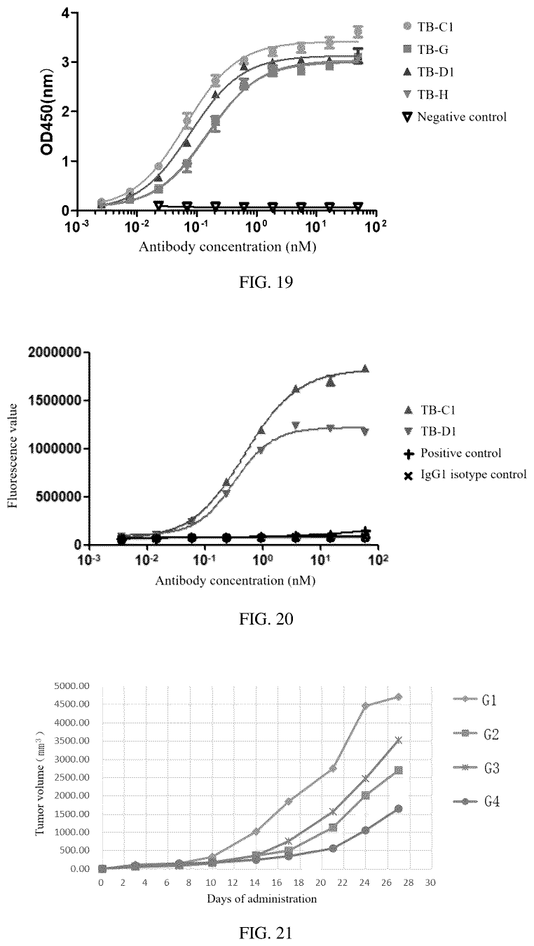

Human TNFR2 mFc (manufacturer: Kaixia, CAT: TN-HM3R2, LOT: 031202) was diluted to 0.3 μg/mL with a coating solution (1×PBS, pH7.4) and coated into a 96-well ELISA plate at 100 μL/well at 4° C. overnight. The coating solution was poured off. Each well was washed with 1×PBST at 300 μL, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. A negative control was set up, which was commercially available trastuzumab for injection (Herceptin). Starting at 50 nM, which was used as the initial concentration for 3-fold dilution, a total of 10 gradients were diluted, and a blank well was set up and only the diluent was added. It was incubated at 37° C. for 1 h at 100 μL/well. The liquid in the well was discarded, washed 4 times with a plate washer, and patted dry on a flat paper. Human 4-1BB His (manufacturer: Acro, CAT: 41B-H5258, LOT: 198-2146F1-W6) was diluted to 0.3 μg/mL, 100 μL was added to each well, and incubated at room temperature for 1 h. The plate was washed 3 times with PBST, and then the HRP labeled His antibody was diluted with sample diluent at 1:5000, 100 μL was added to each well, and incubated at room temperature for 1 h. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader. The ELISA results of antibody molecules are shown in FIG. 18 and FIG. 19 respectively. The results indicate that antibodies TB-A to TB-H have binding activity at both ends.

### Example 6 Detection of the Affinity of Antibodies to TNFR2 Protein by BLI Method (TNFR2+4-1BB)

The antibody sequence variable region of the positive control consists of SEQ ID NO: 25. Soaked the AHC sensor in 0.02% PBST (0.02% Tween 20, pH 7.4, 1×PBS) as a buffer for 600 s to remove the sucrose covering the sensor surface. The AHC sensor was equilibrated with 0.02% PBST (0.02% Tween 20, pH 7.4, 1×PBS) as a buffer for 60 s, the antibodies in the solidified sample plate were equilibrated for 300 s, and the secondary equilibration buffer was equilibrated for 180 s. The 100 nM human TNFR2 His (manufacturer: SINO, CAT: 10417-H08H, LOT: LC15NO0412) protein bind to the antibodies for 300 s and then dissociated for 600 s. After dissociation, 10 mM glycine (pH 2.0) was used as the regeneration buffer and regenerated for 30 s. Regenerating the sensor with 10 mM glycine (pH 2.0). The affinity results of antibody molecules are shown in Table 5. The results indicate that antibody TB-C1 and antibody TB-D1 have affinity for TNFR2 protein.

TABLE 5
Affinity of antibodies for TNFR2 protein

| | KD(M) | kon(1/Ms) | kdis(1/s) |
|---|---|---|---|
| Positive control | 1.61 × 10⁻⁹ | 2.66 × 10⁵ | 4.27 × 10⁻⁴ |
| TB-C1 | 1.82 × 10⁻⁹ | 2.17 × 10⁵ | 3.94 × 10⁻⁴ |
| TB-D1 | 1.28 × 10⁻⁹ | 3.08 × 10⁵ | 3.94 × 10⁻⁴ |

### Example 7 Detection of the Affinity of Antibodies to 4-1BB Protein by BLI Method (TNFR2+4-1BB)

The amino acid sequence of the variable region of the positive control antibody consists of SEQ ID NO: 1 and SEQ ID NO: 5, with the addition of the constant region of human IgG1 (SEQ ID NO: 54 and SEQ ID NO: 55). Soaked the AHC sensor in 0.02% PBST (0.02% Tween 20, pH 7.4, 1×PBS) as a buffer for 600 s to remove the sucrose covering the sensor surface. The AHC sensor was equilibrated with 0.02% PBST (0.02% Tween 20, pH 7.4, 1×PBS) as a buffer for 60 s, the antibodies in the solidified sample plate were equilibrated for 300 s, and the secondary equilibration buffer was equilibrated for 180 s. 100 nM human 4-1BB His protein (manufacturer: Acro, CAT: 41B-H5258, LOT: 198-2146F1-W6) bind to the antibody for 300 s and then dissociated for 600 s. After dissociation, 10 mM glycine (pH 2.0) was used as the regeneration buffer and regenerated for 30 s. Regenerating the sensor with 10 mM glycine (pH 2.0). The affinity results of antibody molecules are shown in Table 6, which indicated that antibody TB-C1 and antibody TB-D1 have affinity for 4-1BB protein.

TABLE 6
Affinity of antibodies for TNFR2 protein

| | KD(M) | kon(1/Ms) | kdis(1/s) |
|---|---|---|---|
| Positive control | 1.42 × 10⁻⁸ | 3.91 × 10⁵ | 5.53 × 10⁻³ |
| TB-C1 | 1.22 × 10⁻⁸ | 5.03 × 10⁵ | 6.16 × 10⁻³ |
| TB-D1 | 1.75 × 10⁻⁸ | 3.69 × 10⁵ | 6.47 × 10⁻³ |

### Example 8 Assay of HEK-293/NFκB-Luci/4-1BB Cell Activation (TNFR2+4-1BB)

The antibody sequence variable region of the positive control consists of SEQ ID NO: 1 and SEQ ID NO: 5, with the addition of the constant region of human IgG1 (SEQ ID NO: 54 and SEQ ID NO: 55). The actual starting concentration and ending concentration of the antibody was 60 nM at 100 μL/well, and diluted by 4-fold gradient (8 gradients in total). HEK-293/NFκB Luci/4-1BB effector cells (from the cell bank of Shenghe (China) Biopharmaceutical Co., Ltd.) were added to the plate at 40 μL/well, and the cell density was 3×10⁴/well; CHOK1-TNFR2 target cells were added to the plate at 40 μL/well, and the cell density was 1×10⁴/well; diluted antibodies were added to the plate at 20 μL/well; after adding the sample, the 96-well full blackboard was placed in a 37° C. incubator for 20 h to incubate; lumiescence fluorescent agent was added at 100 MI/well. The results of the machine testing are shown in FIG. 20. The results indicate that antibody TB-C1 and antibody TB-D1 have an activating effect on HEK-293/NFκB Luci/4-1BB effector cells.

### Example 9 Pharmacodynamic Study of Each Test Substance in the Subcutaneously Transplanted Mouse Colon Cancer Cell Line MC38 Tumor Model in C57BL/6-h4-1BB/hTNFR2 Mice (TNFR2+4-1BB)

6-7 week old female C57BL/6-h4-1BB/hTNFR2 mice (from BioNTech Jiangsu Gene Biotechnology Co., Ltd.) were selected and subcutaneously inoculated them with MC38 tumor cells (from the cell bank of Shenghe (China) Biopharmaceutical Co., Ltd.). After the tumor volume was about 100±50 mm³, they were randomly divided into four groups. Groups include: (1) G1: PBS group; (2) G2: control antibody group, the sequence variable region consists of SEQ ID NO: 1 and SEQ ID NO: 5, with the addition of the constant region of human IgG1 (SEQ ID NO: 54 and SEQ ID NO: 55); (3) G3: antibody TB-C2 group; and (4) G4: antibody TB-D2 group. The negative control group was administered intratumorally with PBS, while the remaining group samples were administered intratumorally with 10 mg/kg. The administration frequency was twice a week, with continuous administration for 4 weeks, administered 8 times in total; the tumor volume and body weight of mice was measured the next day, calculated the tumor volume according to a·b²/2 (a was the long diameter, b was the short diameter). The experimental design was shown in Table 7.

TABLE 7
Experimental scheme design

| | | | Vaccination | | | | Medication |
|---|---|---|---|---|---|---|---|
| | | | amount (per | Test | Dose | Route of | frequency and |
| Group | Mouse | Cell | unit) | drug | (mg/kg) | administration | observation |
| G1 | 4 | MC38 | 5 × 10⁵ | / | / | intraperitoneal | Twice a week |
| | | | | | | injection | |
| G2 | 4 | MC38 | 5 × 10⁵ | Control | 10 | intraperitoneal | Twice a week |
| | | | | antibody | | injection | |
| G3 | 4 | MC38 | 5 × 10⁵ | TB-C2 | 10 | intraperitoneal | Twice a week |
| | | | | | | injection | |
| G4 | 5 | MC38 | 5 × 10⁵ | TB-D2 | 10 | intraperitoneal | Twice a week |
| | | | | | | injection | |

As shown in FIG. 21, antibody TB-D2 has a significant anti-tumor effect.

### Example 10 ADCC Activity of Antibodies Against CHO-4-1BB Cells (TNFR2+4-1BB)

A negative control (IgG1 isotype control) and a reference control (the antibody sequence variable region consists of SEQ ID NO: 1 and SEQ ID NO: 5, with the addition of the constant region of human IgG4, as shown in SEQ ID NO: 55 and SEQ ID NO: 56) were set up. CHO-4-1BB cells overexpressing human 4-1BB (from Hefei Hanke Mabo) were used as target cells, centrifuged at 1000 rpm for 4 min at room temperature, and resuspended in RPMI1640 basic medium (containing 5% FBS). The cells were then seeded on a 96-well plate at 50 μL/well and the cell density was 1×10⁴/well; the antibodies were diluted with RPMI1640 basic culture medium (containing 5% FBS) at an initial concentration of 60 nM, and then diluted by 5-fold gradient (7 gradients in total) at 100 μL/well; resuspended NK cells and added them to the corresponding well at 50 μL/well to make the efficiency target ratio 3:1. At the same time, the maximum target cell lysis well (M), target cell spontaneous release well (ST), effector cell spontaneous release well (SE), total volume correction blank well (BV) and medium blank control well (BM) were set up. After standing for 10 min, it was centrifuged at 1000 rpm at room temperature for 4 min, and incubated in 5% CO₂ and 37° C. carbon dioxide cell incubator for 4 h. L lysate was added into the M and B-V wells 45 min in advance, mixed well, and centrifuged at 1000 rpm at room temperature for 4 min at the end of incubation. 50 μL supernatant was absorbed into the LDH assay plate, and then the substrate dissolved in assay buffer was added at 50 μL/well and reacted at room temperature without light for 30 min. Then termination solution was added at 50 μL/well, and read at 490 nm after standing for 10 min, and calculated the cell mortality rate.

$$cell mortality rate ( \% ) = \frac{{OD}_{Sample hole - ST - SE} \times 100}{{OD}_{M - ST}}$$

Took the logarithm of the antibody concentration as the x-axis, and performed nonlinear regression using Sigmoid dose response (Variable Slope) method (GraphPad Prism software, GraphPad Software, San Diego, California) to obtain the ADCC activity curve of the target antibodies against the target cells.

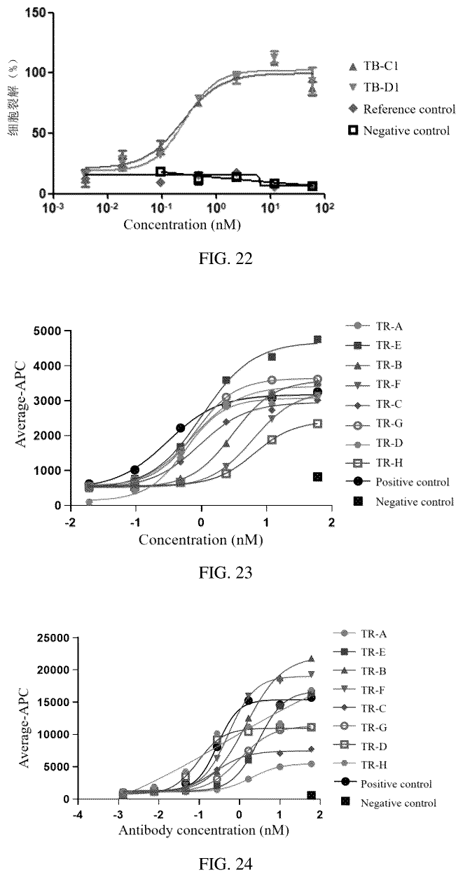

From FIG. 22, it can be seen that antibody TB-C1 and antibody TB-D1 exhibit significant lytic and deathly effects on CHO-4-1BB cells, with a concentration dependence.

### Example 11 Detection of the Binding Activity of Antibodies to CCR8-CHO-K1 Cells by Flow Cytometry (TNFR2+CCR8)

CCR8-CHO-K1 cells overexpressing human CCR8 were constructed using lentiviral transduction of CHO cells. The CCR8 antigen sequence was shown in SEQ ID NO: 57, and the binding activity of the antibody to CCR8-CHO-K1 cells was detected using flow cytometry.

CCR8-CHO-K1 with logarithmic growth and normal morphology was selected and transferred to a centrifuge tube at 1000 rpm for 5 min, resuspended the cells in diluent, and added to a 96-well cell culture plate at 1×10⁵/well. The purified antibodies were diluted to 120 nM with FACS buffer, using this as the starting concentration, and then diluted by 5-fold gradient (6 gradients in total). Negative control and positive control (the variable region of the antibody sequence consists of SEQ ID NO: 9 and SEQ ID NO: 13, and added the constant region of human IgG1, SEQ ID NO: 54 and SEQ ID NO: 55) were set up, and added 100 μL of antibody diluent. The cells were incubated at 4° C. for 60 min, then washed twice with excess FACS buffer. Resuspended the cells in 100 μL FACS Buffer and Added Fluorescent Secondary antibody APC (Biolegend, Cat: 109306) against human IgG FC to the sample. It was incubated for 30 min and washed twice with excess FACS buffer. Resuspended the cells in flow buffer and then detected and analyzed using a flow cytometer. FACS method was used to detect the binding activity between antibodies and CCR8-CHO-K1 cells.

The FACs detection results of antibody binding activity to CCR8-CHO-K1 cells are shown in FIG. 23. Antibodies TR-A to TR-H can specifically bind to CCR8-CHO-K1 cells at multiple concentration ranges, indicating that antibodies TR-A to TR-H have CCR8 binding activity.

### Example 12 Detection of the Binding Activity of Antibodies to TNFR2-CHO-K1 Cells by Flow Cytometry (TNFR2+CCR8)

TNFR2-CHO-K1 cells overexpressing human TNFR2 were constructed using lentiviral transduction of CHO cells. The TNFR2 antigen sequence was shown in SEQ ID NO: 58, and the binding activity of the antibody to TNFR2-CHO-K1 cells was detected using flow cytometry.

TNFR2-CHO-K1 with logarithmic growth and normal morphology was selected and transferred to a centrifuge tube at 1000 rpm for 5 min, resuspended the cells in diluent, and added to a 96-well cell culture plate at 1×10⁵/well. The purified antibodies were diluted to 120 nM with FACS buffer, using this as the starting concentration, and then diluted by 6-fold gradient (7 gradients in total). Irrelated antibody negative control and positive control (antibody sequences consist of SEQ ID NO: 52 and SEQ ID NO: 53, with the addition of the constant region of human IgG1, as shown in SEQ ID NO: 54 and SEQ ID NO: 55) were set up, and added 100 μL of antibody diluent. The cells were incubated at 4° C. for 60 min, then washed twice with excess FACS buffer. Resuspended the cells in 100 μL FACS buffer and added fluorescent secondary antibody APC (Biolegend, Cat: 109306) against human IgG FC to the sample. It was incubated for 30 min and washed twice with excess FACS buffer. Resuspended the cells in flow buffer and then detected and analyzed using a flow cytometer. FACS method was used to detect the binding activity between antibodies and TNFR2-CHO-K1 cells.

The FACs detection results of antibody binding activity to TNFR2-CHO-K1 cells are shown in FIG. 24. Antibodies TR-A to TR-H can specifically bind to TNFR2-CHO-K1 cells at multiple concentration ranges, indicating that antibodies TR-A to TR-H have TNFR2 binding activity.

### Example 13 Dual Target Binding Activity of the Constructed Antibodies (TNFR2+CCR8)

CCR8-CHO-K1 with logarithmic growth and normal morphology (source as above) was selected and transferred to a centrifuge tube at 1000 rpm for 5 min, resuspended the cells in diluent, and added to a 96-well cell culture plate at 1×10⁵/well. The purified antibodies were diluted with FACS buffer to 300 nM, and added 50 μL of human TNFR2 His protein (Yiqiaoshen, CAT: 10417-H08H) diluted to 900 nM to the cells. Negative control 1 (PBS group), negative control 2 (antibody sequence variable region consisting of SEQ ID NO: 9 and SEQ ID NO: 13, with the addition of the constant region of human IgG1, as shown in SEQ ID NO: 54 and SEQ ID NO: 55), and negative control 3 (antibody sequence consisting of SEQ ID NO: 52 and SEQ ID NO: 53, with the addition of the constant region of human IgG1, as shown in SEQ ID NO: 54 and SEQ ID NO: 55) were set up. The cells after adding the sample were incubated at 4° C. for 60 min, and then washed twice with excess FACS buffer. Resuspended the cells in 100 μL FACS buffer and added FITC anti His tag (Abcam, Cat: ab1206) fluorescent secondary antibody carrying FITC to the sample. It was incubated for 30 min and washed twice with excess FACS buffer. Resuspended the cells in flow buffer and then detected and analyzed using a flow cytometer.

The results of detecting the binding of antibodies TR-A to TR-H to CCR8 and TNFR2 using FACS method are shown in Table 8. Compared with the negative control, antibodies TR-A to TR-H have binding activity at both ends, indicating that antibodies TR-A to TR-H can simultaneously bind to CCR8 and TNFR2, antibodies TR-E and TR-C have the best binding activity at both ends.

TABLE 8
Average fluorescence values of antibody binding at both ends

| Antibodies | Average FITC-A |
|---|---|
| TR-A | 39964 |
| TR-E | 61880 |
| TR-B | 28988 |
| TR-F | 27621 |
| TR-C | 89497 |
| TR-G | 33680 |
| TR-D | 30329 |
| TR-H | 34156 |
| Negative control 1 (PBS) | 15212 |
| Negative control 2 (CCR8) | 22967 |
| Negative control 3 (TNFR2) | 22563 |

### Example 14 ADCC Activity of Antibodies Against CCR8-CHO-K1 Cells (TNFR2+CCR8)

Negative control (IgG1 isotype control) and positive control (antibody sequence variable region consisting of SEQ ID NO: 9 and SEQ ID NO: 13, with the addition of the constant region of human IgG1, as shown in SEQ ID NO: 54 and SEQ ID NO: 55) were set up. CCR8-CHO-K1 cells overexpressing human CCR8 (from the same source as above) was used as target cells, centrifuged at 1000 rpm for 4 min at room temperature and resuspended in RPMI1640 basic medium (containing 5% FBS), then spread on a 96-well plate at 50 μL/well and the cell density was 1×10⁴/well. The antibodies were diluted with RPMI1640 basic culture medium (containing 5% FBS) at 60 μg/mL as the starting concentration, and then diluted by 10-fold gradient (7 gradients in total) at 100 μL/well; resuspended NK cells and added them to the corresponding well at 50 μL/well to make the efficiency target ratio 3:1. At the same time, the maximum target cell lysis well (M), target cell spontaneous release well (ST), effector cell spontaneous release well (SE), total volume correction blank well (BV) and medium blank control well (BM) were set up. After standing for 10 min, it was centrifuged at 1000 rpm at room temperature for 4 min, and incubated in 5% C02 and 37° C. carbon dioxide cell incubator for 4 h. Lysate was added into the M and B-V wells 45 min in advance, mixed well, and centrifuged at 1000 rpm at room temperature for 4 min at the end of incubation. 50 μL supernatant was absorbed into the LDH assay plate, and then the substrate dissolved in assay buffer was added at 50 μL/well and reacted at room temperature without light for 30 min. Then termination solution was added at 50 μL/well, and read at 490 nm after standing for 10 min, and calculated the cell mortality rate.

$$cell mortality rate ( \% ) = \frac{{OD}_{Sample hole - ST - SE} \times 100}{{OD}_{M - ST}}$$

Took the logarithm of the antibody concentration as the x-axis, and perform nonlinear regression using Sigmoid dose response (Variable Slope) method (GraphPad Prism software, GraphPad Software, San Diego, California) to obtain the ADCC activity curve of the target antibody against the target cells.

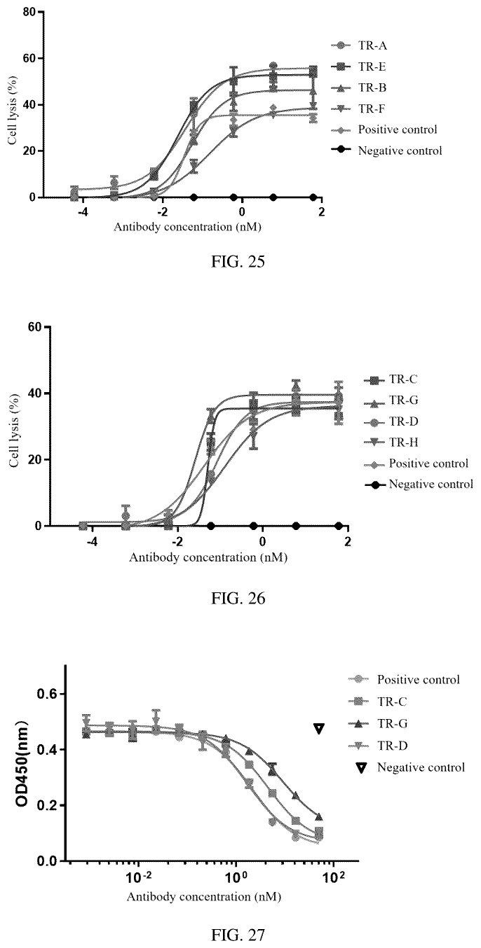

From FIG. 25 and FIG. 26, it can be seen that the negative control do not show any killing effect on CCR8-CHO-K1 cells, while the positive control and antibodies TR-A to TR-H show concentration dependent lysis and death of CCR8-CHO-K1 cells.

### Example 15 Determination of the Blocking Activity of Antibodies to TNFR2 by ELSIA (TNFR2+CCR8)

Human TNFR2 mFC (KaKa, CAT: TNF-HM3R2) was diluted to 3.5 μg/mL with a coating solution (1×PBS, pH 7.4), added into a 96-well enzyme-linked immunosorbent assay plate at 100 μL/well, and coated overnight at 4° C. The coating solution was poured off. Each well was washed with 1×PBST at 300 μL, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. Negative control and positive control were set up. The negative control was commercially available injectable trastuzumab (Herceptin), and the variable region of the positive control antibody sequence consists of SEQ ID NO: 25. The positive control and antibody were diluted diluted to 100 nM with 3% skim milk powder as the starting concentration, and then diluted by 3-fold gradient (11 gradients in total), and a blank well was set up and only the diluent was added. The antibodies and human TNF alpha His protein (novoprotein, Cat: C008) were diluted to 1 μg/mL, added into an enzyme-linked immunosorbent assay (ELISA) plate coated with human TNFR2 mFC protein at 50 μL/well, and incubated at 37° C. for 1 h. The liquid in the well was discarded, washed 4 times with a plate washer, and patted dry on a flat paper. The anti his antibody (Abcam, Cat: ab1187) was diluted with 3% skim milk powder at 1:5000, 100 μL was added to each well, and incubated at 37° C. for 1 h. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader.

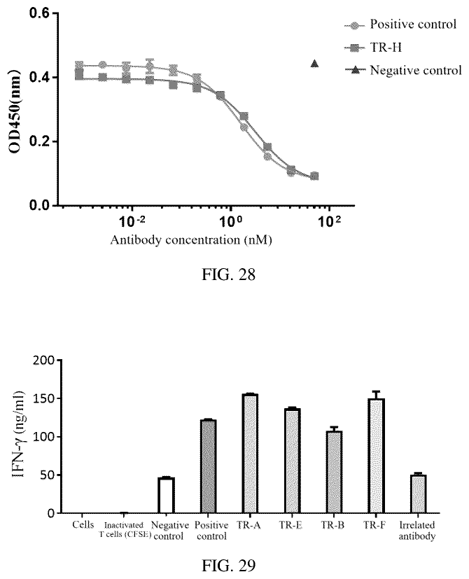

The ELISA results of antibody molecules blocking TNFR2-TNF are shown in FIG. 27 and FIG. 28. The results indicate that antibodies TR-C, TR-D, TR-G, and TR-H can all block the binding of TNFR2 to TNF.

### Example 16 Assay of CD8+T Cell Activation (TNFR2+CCR8)

The antibodies were diluted to 40 μg/mL (final experimental concentration of 20 g/mL, 50 μL/well) with PBS; and OKT3 was diluted to 10 μg/mL (final experimental concentration of 5 μg/mL, 50 μL/well) with PBS. Irrelated antibody, which was human IgG1 kappa Isotype control (HG1K, Sino Biological) was set up. A negative control and a positive control were set up. The variable region of the antibody sequence in the positive control consists of SEQ ID NO: 52 and SEQ ID NO: 53, with the addition of the constant region of human IgG1 (SEQ ID NO: 54 and SEQ ID NO: 55). 50 μL of OKT3 dilution solution and 50 μL of antibody dilution solution were coated into a 96-well flat plate, with a total volume of 100 μL/well, and incubated overnight at 4° C. On the second day, CD8+T cells were negatively isolated from frozen PBMCs (ORiCELLS, ID: PCH20201200031), and purity testing was performed on the sorted negative CD8+T cells; CD8+T cells were labeled with CFSE at a concentration of 5 μM, and incubated at 37° C. for 10 min, and terminated staining by adding complete culture medium. The plate used for coating was washed once with PBS, and CFSE labeled or unlabeled T cells were added at 100 μL/well and the cell density was 2×10⁵/well, and anti-CD28 at a final concentration of 1 μg/mL was added to 100 μL/well. The total experimental system was 200 μL/well. It was incubated at 37° C. for 96 h; after centrifugation, the supernatant was collected for the detection of IFN-7 and IL2 cytokines. The cells were washed with PBS for flow cytometry analysis. As shown in FIG. 29, antibodies TR-A, TR-E, TR-B, and TR-F all have an activating effect on CD8+T cells, and compared with the negative control, they have the ability to promote the release of more IFN-γ from CD8+T cells.

The protected content of the present invention is not limited to the above embodiments. All variations and advantages that can be conceived by those skilled in the art without departing from the spirit and scope of the inventive concept are included in the present invention and are protected by the appended claims.

1. A bispecific antibody, comprising: (a) a first antibody or antigen-binding fragment thereof that specifically binds to a first antigen; and (b) a second antibody or antigen-binding fragment thereof that specifically binds to a second antigen; wherein the first antigen is a T cell immunomodulator, and the second antigen is TNFR2.

2. The bispecific antibody of claim 1, wherein the T cell immunomodulator is a T cell co-stimulatory immunomodulator or a T cell co-inhibitory immunomodulator.

3. The bispecific antibody of claim 2, wherein the first antibody or antigen-binding fragment thereof comprises a heavy chain and a light chain; and the second antibody or antigen-binding fragment thereof comprises scFv or VHH.

4. The bispecific antibody of claim 3, wherein the heavy chain variable region of one heavy chain and the light chain variable region of one light chain of the first antibody form an antigen-binding site, and the heavy chain variable region of another heavy chain and the light chain variable region of another light chain form an antigen-binding site.

5. The bispecific antibody of claim 4, further comprising one first antibody or antigen-binding fragment thereof and one or more scFv or VHH.

6. The bispecific antibody of claim 5, further comprising one first antibody or antigen-binding fragment thereof and one scFv or VHH, wherein the scFv or VHH is connected to the N-terminus or C-terminus of the heavy chain of the first antibody or antigen-binding fragment thereof.

7. The bispecific antibody of claim 5, further comprising one first antibody or antigen-binding fragment thereof and two scFvs or two VHHs.

8. The bispecific antibody of claim 7, wherein the two scFvs or two VHHs are respectively connected to the N-terminus or C-terminus of two heavy chains of the first antibody or antigen-binding fragment thereof.

9. The bispecific antibody of claim 8, further comprising two first polypeptide chains and two second polypeptide chains, wherein for each of the polypeptide chains: (a) the first polypeptide chain independently comprises the light chain of the first antibody or antigen-binding fragment thereof; and (b) the second polypeptide chain independently comprises the heavy chain of the first antibody or antigen-binding fragment thereof and the scFv or VHH.

10. The bispecific antibody of claim 9, wherein the two first polypeptide chains are identical or different, and/or the two second polypeptide chains are identical or different.

11. The bispecific antibody of claim 10, wherein the T cell co-stimulatory immunomodulator is selected from the group consisting of: 4-1BB, OX40, B7H2, GITR, CD48, HVEM, Nectin-2, CD40, CD30, CD28, CD27, B7-1, B7-2, LIGHT, B7H6, 2B4, CD28H or NKp30.

12. The bispecific antibody of claim 10, wherein the T cell co-inhibitory immunomodulator is selected from the group consisting of: CCR8, PD-1, PD-L1, PD-L2, B7H3, B7H4, B7H5, BLTA, CD96, CD47, CD155, CTLA-4, LAG-3, TIGIT, TIM-3, CD111, DNAM-1, Galectin-9, Nectin-3, PVRIG, SIRP α, SIRP β, SIRP α V2 or CD160.

13. The bispecific antibody of claim 12, wherein the heavy chain variable region and the light chain variable region of the scFv are connected by a linker L1.

14. The bispecific antibody of claim 13, wherein the scFv or VHH is connected to the N-terminus or C-terminus of the heavy chain of the first antibody or antigen-binding fragment thereof by a linker L2.

15. The bispecific antibody of claim 14, wherein the linker L1 and linker L2 are identical or different.

16. The bispecific antibody of claim 15, wherein the linker L1 and/or linker L2 has an amino acid sequence as shown in (G4S)ₓ, where x is an integer selected from 1-6.

17. The bispecific antibody of claim 16, wherein the heavy chain of the first antibody or antigen-binding fragment thereof comprises a heavy chain variable region and a heavy chain constant region, and the light chain comprises a light chain variable region and a light chain constant region.

18. The bispecific antibody of claim 17, wherein the heavy chain of the first antibody or antigen-binding fragment thereof comprises a first Fc region and a second Fc region.

19. The bispecific antibody of claim 18, wherein the first Fc region and the second Fc region are identical or different.

20. The bispecific antibody of claim 19, wherein the first Fc region or the second Fc region is selected from IgG, IgA, IgD, IgE, IgM, or variants thereof.

21-48. (canceled)
